(19) United States
(12) Patent Application Publication
(10) Pub. No.: US 2004/0166060 A1
(43) Pub. Date: Aug. 26, 2004

(54) Liposomal encapsulation of alpha particle emittors and uses thereof

(75) Inventors: George Sgouros, Ellicott City, MD (US); James L. Thomas, Cedar Crest, NM (US); David A. Scheinberg, New York, NY (US); Michael R. McDevitt, Bronx, NY (US); Stavroula Sofou, New York, NY (US)

Correspondence Address: Benjamin Aaron Adler, ADLER & ASSOCIATES, 8011 Candle Lane, Houston, TX, 77071, US

(73) Assignee: Sloan Kettering Institute for Cancer Research

(21) Appl. No.: 10/720,904

(22) Filed: Nov. 24, 2003

Related U.S. Application Data
(63) Continuation-in-part of application No. 10/319,978, filed on Dec. 16, 2002.
Application No. 10/319,978 is a continuation-in-part of application No. PCT/US01/19133, filed on Jun. 15, 2001.
(60) Provisional application No. 60/212,186, filed on Jun. 16, 2000.

(51) Int. Cl. A61M 36/14; A61K 51/00

(52) U.S. Cl. 424/1.65

(57) ABSTRACT

The present invention provides targeted delivery of alpha particle-emitting radionuclides and their alpha-emitting progeny using liposomal encapsulation to reduce the loss of daughter radionuclides from the targeting vehicle and, therefore, from the tumor site. Also provided are encapsulated alpha particle-emitting radionuclides.

### CROSS-REFERENCE TO RELATED APPLICATION

This application is a continuation-in-part of national stage application U.S. Ser. No. 10/319,978, filed Dec. 16, 2002, which is a continuation-in-part of international application PCT/US01/19133, filed Jun. 15, 2001, now abandoned, which claims benefit of provisional patent application U.S. Serial No. 60/212,186, filed Jun. 16, 2000, now abandoned.

### FEDERAL FUNDING LEGEND

This invention was produced in part using funds obtained through grants DAMD170010657 from the USAMRMC and R01 CA55349 from the NIH. Consequently, the federal government has certain rights in this invention.

### BACKGROUND OF THE INVENTION

1. Field of the Invention

The present invention relates generally to the field of radiotherapy. More specifically, the present invention relates to liposomal encapsulation of alpha particle-emitting radionuclides. Most specifically, the present invention relates to liposomal encapsulation of alpha-particle emittors and uses thereof.

2. Description of the Related Art

Optimal treatment with many drugs requires maintenance of a drug level for an extended period of time. For example, optimal anti-cancer treatment with cell cycle-specific antimetabolites requires maintenance of a cytotoxic drug level for a prolonged period of time. The half-life of many drugs after an intravenous (IV), subcutaneous (SC), intraperitoneal (IP), intraarterial (IA), intramuscular (IM), intrathecal (IT), or epidural dose is very short, being in the range of a fraction of an hour to a few hours.

Epithelial ovarian cancer is described as the “silent killer” because in the majority (70%) of patients the disease is first detected as a result of symptoms arising after the disease has spread outside of the pelvis and into the peritoneal cavity. In such cases of advanced disease (FIGO-Stage III), the 5-year survival rate employing current treatment approaches is approximately 15 to 20%. Disseminated, metastatic cancer is essentially incurable and disseminated ovarian carcinoma is rarely cured by current treatment options. Thus, a new treatment modality is needed for disseminated epithelial ovarian carcinoma or other cancers.

Alpha particle-emitting radionuclides are highly potent cytotoxic agents capable of tumor-cell kill without limiting morbidity and, as such, hold great promise as potential therapeutic agents for disseminated disease such as in cancer treatment. Due to their high energy deposition per distance traveled, alphas are capable of sterilizing individual cells or cell clusters with only one to three traversals through the nucleus. In addition, the 50-100 μm range of alphas is consistent with the dimensions of disseminated disease, allowing for localized irradiation of target cells with minimal normal cell irradiation.

The radionuclides, astatine-211 (At-211) and bismuth-213 (Bi-213) have been investigated clinically (1-3). Both have short half-lives, 7 hours and 46 minutes, respectively, and are, therefore, appropriate for situations in which targeting is very rapid. The first injection of an alpha-particle emitter to humans for radioimmunotherapy (RIT) was with ²¹³Bi conjugated to the anti-CD33 antibody, HuM195, targeting myeloid leukemia (1). This trial demonstrated feasibility and anti-cancer activity with minimal toxicity. In the second of these human trials the anti-tenascin antibody, 81C6, labeled with the alpha-particle emitter ² lAt, was injected into surgically created cavities in the patients with malignant gliomas. This trial has demonstrated substantially better tumor control relative to ¹³¹I-labeled 81C6 antibody (3). Animal studies have shown that alpha-particle emitters yield superior tumor control relative to beta or Auger electron emitters (4-9).

In treating dissemated diseases or cancer, e.g., late-stage breast cancer with measurable liver or bone metastases, longer-lived alpha particle emitters are required to reach distant metastases that have developed their own vasculature. The effectiveness of alpha particles arises because the amount of energy deposited per unit distance traveled, the linear energy transfer or LET, is approximately 400 times greater than that of beta particles, i.e., 80 keV/μm vs. 0.2 keV/μm. Cell survival studies have shown that alpha-particle induced killing is independent of oxygenation state or cell-cycle during irradiation and that as few as 1 to 3 tracks across the nucleus may result in cell death (10-12).

One of the most promising alpha-particle emitting radionuclides, Ac-225 has a 10-day half-life and, therefore, unlike ²¹³Bi (45.6-min half-life) and ²¹¹At (7.2 h half-life) is not limited to the targeting of rapidly accessible disease. ²²⁵Ac (t_{1/2}=10 days) decays to three daughters, i.e., α-emitting radionuclides ²²¹Fr (t_{1/2}=4.9 min), ²¹⁷At (t_{1/2}=32 msec) and ²¹³Bi (45.6 min) (FIG. 1). Studies, in vitro, have shown that this radionuclide is approximately 1000-fold more effective than the first generation alpha-emitter, ²¹³Bi that is currently under clinical investigation. Studies in animals, however, have also shown that it is substantially more toxic. The increased efficacy and toxicity are a result of the alpha-particle emitting intermediates. When these are confined to the target cells, efficacy is increased, when they distribute throughout the body, toxicity is increased (13).

This toxic potential presents a fundamental difficulty if antibody or other molecular approaches to delivery of this radionuclide are used since the bond between the targeting vehicle and the radionuclide is broken upon transformation of the parent and emission of the first alpha. This leaves the first daughter in the decay chain free to distribute throughout the body where it will decay and subsequently yield additional alpha emissions to normal organs from subsequent daughter decays. Of the 4 alphas, only the first one originating from decay of Ac-225 contributes to the tumor dose, the remainder will distribute throughout normal tissue to increase toxicity. The radiotoxicity of the Ac-225 daughter isotopes is a limiting factor in radioimmunological therapies. This problem is lessened if the daughter radioisotope can be retained within a delivery vehicle (14).

Liposomes and their potential as drug-delivery vehicles have been investigated for many years. Liposomes are structures defined by a phospholipid bilayer membrane that encloses an aqueous compartment (15-16). The membrane acts as a barrier that inhibits free molecular diffusion across the bilayer. Multivesicular liposomes (MVL) (17) are uniquely different from other lipid-based drug delivery systems such as unilamellar (18-19) and multilamellar (20) liposomes. In contrast to unilamellar liposomes (also known as unilamellar vesicles, or “ULV”), multilamellar and multivesicular liposomes (MVL) contain multiple aqueous chambers per particle. Because of the similarity in acronyms, multivesicular liposomes (MVL) are frequently confused with multilamellar liposomes (MLV). Nevertheless, the two entities are entirely distinct from each other. Whereas multilamellar liposomes (also known as multilamellar vesicles or MLV) contain multiple concentric chambers within each liposome particle, resembling the “layers of an onion,” the multiple aqueous chambers in multivesicular liposomes are non-concentric. The structural differences between unilamellar, multilamellar, and multivesicular liposomes are well known in the art.

The structural and functional characteristics of multivesicular liposomes are not directly predictable from current knowledge of ULV and multilamellar liposomes. The differences are described in the book (21). Multivesicular liposomes are bounded by an external bilayer membrane shell, but have a very distinctive internal morphology, which may arise as a result of the special method employed in the manufacture. Topologically, multivesicular liposomes (MVL) are defined as liposomes containing multiple non-concentric chambers within each liposome particle, resembling a “foam-like” matrix. The presence of internal membranes distributed as a network throughout multivesicular liposomes may serve to confer increased mechanical strength to the vesicle, while still maintaining a high volume:lipid ratio as compared to multilamellar liposomes.

The multivesicular nature of multivesicular liposomes also indicates that, unlike in unilamellar vesicles, a single breach in the external membrane of a multivesicular liposome will not result in total release of the internal aqueous contents. Thus, both structurally and functionally the multivesicular liposomes are unusual, novel and distinct from all other types of liposomes. As a result, the functional properties of multivesicular liposomes are not predictable based on the prior art related to conventional liposomes such as unilamellar vesicles and multivesicular liposomes.

The prior art. for example, U.S. Pat. Nos. 4,522,803; 4,310,506; 4,235,871; 4,224,179; 4,078,052; 4,394,372; 4,308,166; 4,485,054; and 4,508,703, describes a number of techniques for producing unilamellar vesicles and multivesicular liposomes. The prior art also describes methods for producing multivesicular liposomes (17, 22). In one method (17), the pharmaceutical utility of multivesicular liposomes encapsulating small therapeutic molecules, such as cytosine arabinoside or cytarabine, is limited. Subsequent studies (23) showed that the release rate of encapsulated molecules into biological fluids can be modulated by encapsulating in the presence of a hydrochloride.

Although liposomes have been used in the delivery of chemotherapy and in gene targeting (16, 24), the use of liposomes in the delivery of radioactivity has not been accepted. Their application in drug delivery has been made possible by the development of sterically stabilized structures that use polyethylene glycol (PEG) chains or monosialogangliosides to reduce uptake and catabolism of IV-administered liposomes by the reticuloendothelial system (RES), thereby increasing circulatory half-life. Such liposomes are typically 100 to 150 nm in diameter since this size range reduces RES uptake while retaining adequate aqueous volume for drug delivery. Liposome tumor localization is dependent on the differential permeability of normal vs tumor tissue capillaries (23-25).

Liposomes have also been used to deliver radionuclides, primarily for tumor diagnosis and infectious site imaging (26-27). The use of such liposomes for the delivery of actinium-225 (Ac-225) and other promising alpha emitters, such as radium-223 (Ra-223) is particularly compelling because they may retain daughter radionuclides within the aqueous phase thereby reducing systemic toxicity. Since range of an alpha particle (50-100 microns) is sufficient to penetrate beyond the liposomal membranes (70 nm) and alpha-particles traverse the phospholipid membrane without energy dissipation, tumor irradiation will be enhanced.

Studies in animals have shown that ²²⁵Ac is substantially more potent and more toxic than ²¹³Bi (12, 21, 22) (13, 28). The toxicity arises because conjugation to antibodies or other molecular vehicles for targeting of this radionuclide can deliver stably only the first of the four alpha particle emitting atoms. The bond between the targeting vehicle and the chelate holding the radionuclide may be broken upon transformation of the parent to new daughter and emission of the first alpha particle, the new daughter has different chemistry than the parent, or there is recoil of the parent atom away from the chelate. Subsequent alpha-particle emitting daughters are, therefore, free to distribute throughout the body and to irradiate healthy organs and tissues.

Liposomes with encapsulated ²²⁵Ac should also retain the alpha-emitting intermediates at the tumor site. Owing to their position in the periodic table, the daughters of ²²⁵Ac are likely to be ionic (Fr⁺¹, At⁻¹, Bi⁺). Binding or association of inorganic monovalent ions to non-charged hydrophilic surfaces, like phospholipid membranes, has been treated theoretically (31) and verified experimentally (32). In addition, certain anions have the tendency to associate with phosphatidylcholine lipids (31). The hydrophobic region of liposomal membranes provides a barrier to loss by diffusion of ionic species entrapped in the internal aqeuous compartment (33). Therefore, if ²²⁵Ac is initially entrapped within the aqueous compartment of liposomes, the daughters are expected to be retained within the liposomes. Some loss is unavoidable, however, because the recoil distance traveled by daughter atoms, upon decay of the parent, is of the order of the liposome size.

Liposomes may be used for locoregional and intracavitary therapy of disseminated micrometastases (34). Liposomes of large size are rapidly cleared from blood circulation (35). Targeted delivery may be achieved with appropriate antibodies attached at the terminal end of the PEG-chains; this geometry provides exposed antibody molecules that protrude from the liposome for unhindered antigen recognition. Similarly, liposomes have been shown to achieve increased cellular uptake by receptor-mediated endocytosis (36).

The inventors have recognized a need in the art for an method of targeted delivery of alpha particle emitting radionuclides with improved retention of the daughter radionuclides within the delivery vehicle. Liposomal encapsulation presents a strategy for the delivery of actinium to and retention of the daughters at the tumor site. The prior art is deficient in an effective means for sequestering Ac-225 and its daughter radionuclides at specific targets during radiotherapy. The present invention fulfills this long-standing need and desire in the art.

### SUMMARY OF THE INVENTION

The present invention is directed to a method of reducing the systemic release of radioactive decay intermediates upon administration of an alpha particle-emitting radionuclide to an individual. The radionuclide is incorporated into large liposomes having a diameter sufficient to retain at least a majority of the radioactive decay intermediates produced by the radionuclide. The large liposomes are administered to the individual such that retention within the large liposomes of the radioactive decay intermediates of the radionuclide reduces the systemic release thereof.

The present invention also is directed to a method of targeting cells in an individual for liposomal delivery of an alpha particle-emitting radionuclide thereto with reduced systemic release of radioactive decay intermediates whereby the radionuclide is entrapped within small liposomal vesicles. The entrapped radionuclide is incorporated into the aqueous phase of large liposomes having a diameter sufficient to retain at least a majority of the radioactive decay intermediates produced by the radionuclide. The large liposome comprises polyethyleneglycol-linked lipids (PEG-lipids) on the outer membranes thereof and targeting agents specific to the cells attached to the PEG-lipids. The radionuclide is delivered to the cell whereby the targeting agents target the cells whereby retention within the large liposomes of the majority of the radioactive decay intermediates of the radionuclide reduces the systemic release thereof.

The present invention is directed further to a method of targeting cancer cells expressing HER-2/neu protein in an individual for liposomal delivery of Ac-225 thereto with reduced systemic release of radioactive decay intermediates. The Ac-225 is entrapped within small liposomal vesicles which is incorporated into the aqueous phase of large liposomes having a diameter sufficient to retain at least a majority of the radioactive decay intermediates of Ac-225. The large liposome comprises polyethyleneglycol-linked lipids (PEG-lipids) on the outer membranes thereof and Herceptin antibodies attached to the PEG-lipids. The Ac-225 is delivered to the cell such that the targeting agents target the cells while retention within the large liposomes of the majority of the radioactive decay intermediates of the radionuclide reduces the systemic release thereof.

The present invention is directed further yet to an encapsulated alpha particle-emitting radionuclide. The encapsulated radionuclide comprises the alpha particle emitting radionuclide, small liposome vesicles entrapping the alpha particle-emitting radionuclide and a large liposome incorporating the small liposome vesicles. The large liposome has a diameter sufficient to retain at least a majority of the radioactive decay intermediates of the alpha particle-emitting radionuclide.

Other and further aspects, features, and advantages of the present invention will be apparent from the following description of the embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate embodiments of the invention and therefore are not to be considered limiting in their scope.

FIG. 1 depicts the actinium-225 decay cascade with associated particulate decays and half lives.

FIGS. 2A-2B depict negative staining TEM images of 800 nm (FIG. 2A) and 100 nm (FIG. 2B) in filter diameter liposomes.

FIG. 3 shows the radioactivity collected in each fraction after Sephadex™ column chromatography at different times after ¹¹¹In encapsulation.

FIG. 4 shows the retention of ¹¹¹In in liposomes as a function of time after loading.

FIGS. 5A-5B demonstrate that calcein-containing liposomes remain intact in the peritoneum. Calcein fluorescence in the peritoneum is minimal prior to the addition of Triton-X 100 in both zwitterionic liposomes (FIG. 5A) and cationic liposomes (FIG. 5B).

FIG. 6 shows the model of Ac-225 radioactive decay used to determine loss rate of daughter radionuclides from liposome-encapsulated Ac-225 activity.

FIGS. 7A and 7B show sample simulations using models of the transfer rate from each sub-compartment within liposomes to the extraliposomal compartment.

FIGS. 8A-9D depict simulations obtained using four different loss rates.

FIGS. 9A and 9B show the relationship between loss rate and the levels of daughter activity at different measurement times after liposome separation.

FIGS. 10A-10C depict the biodistribution of ¹¹¹In-DTPA only (FIG. 10A) zwitterionic liposomes (FIG. 10B), ¹¹¹In-DTPA cationic liposomes (FIG. 10C).

FIG. 11 depicts the theoretical predictions of ²¹³Bi retention for different liposome sizes (solid line). Radionuclides were assumed to be uniformly entrapped within the liposomal aqueous compartment. The average recoil distance of 87.6 nm was used for all alpha-emitting intermediates to simplify calculations. Binding of radionuclides to the liposomal membrane will significantly reduce retention (dotted line).

FIG. 12A depicts actinium-225 retention for 100 nm (●), 400 nm (◯) and 800 (▾) nm zwitterionic liposomes over a period of 30 days. The error bars correspond to standard errors of repeated measurements.

FIG. 12B depicts actinium-225 retention for 100 nm (●), 400 nm (◯) and 800 (▾) nm positively charged liposomes over a period of 30 days. The error bars correspond to standard errors of repeated measurements.

FIG. 13A depicts bismuth-213 retention for 100 (●), 400 nm (◯) and 800 (▾) nm zwitterionic liposomes over a period of 30 days. The error bars correspond to standard errors of repeated measurements.

FIG. 13B depicts bismuth-213 retention for 100 (●), 400 nm (◯) and 800 (▾) nm cationic liposomes over a period of 30 days. The error bars correspond to standard errors of repeated measurements.

FIG. 14A is a diagram of the preparation of multivesicular liposomes. I: small biotin-labeled liposomes loaded with actinium-225 (not shown) are prepared; II.: small liposomes are entrapped within large liposomes to yield MUVELs; III: unentrapped small liposomes are separated from MUVELs; and IV: MUVELs containing large liposomes comprising in part the biotin-labeled small liposomes are removed with a strepavidin affinity colum.

FIG. 14 depicts a cryo TEM image of MUVEL suspension.

FIG. 15A depicts calcein quenching ratios for each eluted fraction in MUVELs. Calcein was entrapped at self-quenching concentrations only in the small liposomes (open symbols).

FIG. 15B depicts total entrapped calcein concentration after addition of Triton x-100 on each MUVEL fraction from S-1000 (Day 1: open symbols. Day 4: closed symbols).

FIG. 16 depicts calcein quenching ratios of fractions 15, 16, 17, 18 from S-1000 over four days.

FIG. 17 depicts the parallel elution profiles of large (800 nm filter diameter-closed symbols) and sonicated (open symbols) liposomes.

FIG. 18A depicts the percentage of actinium retention in small liposomes (●), large liposomes (▪) and MUVELs (▾).

FIG. 18B depicts the percentage of bismuth retention in small liposomes (●), large liposomes (▪) and MUVELs (▾).

FIG. 19 is optical images of the peritoneum shown ventral view, head up eighteen hours after intraperitoneal injection of FITC-Herceptin. The perisplenic tumor (T), microscopic tumor nodules (arrows) and urine in the bladder (B), which autofluoresces, are seen on the fluorescent image in panel 2. Corresponding regions are identified on the bright field image in panel 1. This approach will be useful in evaluating the relative localization of Herceptin Ab vs. the Herceptin-coated immunoliposomes.

### DETAILED DESCRIPTION OF THE INVENTION

In one embodiment of the present invention there is provided a method of reducing the systemic release of radioactive decay intermediates upon administration of an alpha particle-emitting radionuclide to an individual, comprising the steps of incorporating the radionuclide into large liposomes having a diameter sufficient to retain at least a majority of the radioactive decay intermediates and administering the large liposomes to the individual wherein retention within said large liposomes of said radioactive decay intermediates produced by said radionuclide reduces the systemic release thereof.

In one aspect of this embodiment the method further comprises the step of entrapping the radionuclide within smaller liposomal vesicles prior to incorporating the radionuclide into the aqueous phase of the larger liposome. In a related aspect the smaller liposomal vesicles are labeled with biotin. In another aspect of this embodiment the method further comprises preinjecting the individual with empty large liposomes and saturating the reticuloendothelial organs to reduce non-tumor specific spleen and liver uptake of the radionuclide upon adminstration thereof.

In yet another aspect of this embodiment the method further comprises coating the outer membrane surfaces of the large liposomes with molecules that preferentially associate with a specific target cell. These molecules or targeting agents may be antibodies, peptides, engineered molecules or fragments thereof. A representative example of such an antibody is Herceptin. The target cells may be a cancer cell, a virally infected cell, an autoimmune cell, or an inflammatory cell. Examples of such cancer cells are those found in ovarian cancer, breast cancer or metastatic cells thereof.

In all aspects of this embodiment the large liposomes have a diameter of about 600 nm to about 1000 nm. The large liposomes may further comprise molecules incorporated into the outer membranes to stabilize them. An example of such a molecules are polyethyleneglycol-linked lipids (PEG-lipids). These molecules may be used to attach to a targeting molecule as disclosed supra. The large liposomes disclosed herein may further comprise stabilizing agents or have an aqueous phase with a high pH. Representative examples of stabilizing agents are a phosphate buffer, an insoluble metal binding polymer, resin beads, metal-binding molecules or halogen binding molecules incorporated into the aqueous phase to further facilitate retention of the radioactive decay intermediates. Additionally, the large liposomes may comprise molecules to facilitate membrane fusion with the target cells or to facilitate endocytosis by the target cells.

Again in all aspects of this embodiment the alpha particle emitting radionuclide may be incorporated into the aqueous phase as a chelation compound with or without other stabilizing agents. Representative examples of this radionuclide are ²²⁵Ac, ²²³Ra, ²¹³Bi or ²¹¹At. A preferred radionuclide is ²²⁵Ac. Alternatively, the alpha emitting radionuclide itself may be a daughter of a beta emitting radionuclide which is incorporated within the large liposomes. An example of a beta emitting radionuclide is ²¹²Pb.

In another embodiment of the present invention there is provided a method of targeting cells in an individual for liposomal delivery of an alpha particle-emitting radionuclide thereto with reduced systemic release of radioactive decay intermediates comprising the steps of encapsulating the radionuclide within small liposomal vesicles; incorporating the radionuclide into the aqueous phase of large liposomes having a diameter sufficient to retain at least a majority of the radioactive decay intermediates of said radionuclide. The liposome comprises polyethyleneglycol-linked lipids (PEG-lipids) on the outer membranes thereof and targeting agents specific to the cells attached to the PEG-lipids. The radionuclide is delivered to the cell whereby the targeting agents target the cells while retention within said large liposomes of said radioactive decay intermediates produced by said radionuclide reduces the systemic release thereof. In all aspects of this embodiment further method steps, the radionuclides, the antibodies, targeting agents, the liposomes and the components thereof are as described supra.

In yet another embodiment of the present invention there is provided a method of targeting cancer cells expressing HER-2/neu protein in an individual for liposomal delivery of Ac-225 thereto with reduced systemic release of radioactive decay intermediates thereof comprising the steps of entrapping the Ac-225 within small liposomal vesicles; incorporating the entrapped Ac-225 into the aqueous phase of large liposomes having a diameter sufficient to retain at least a majority of the radioactive decay intermediates of Ac-225 where the large liposome comprises polyethyleneglycol-linked lipids (PEG-lipids) incorporated into outer membranes thereof and Herceptin antibodies attached to the PEG-lipids; and delivering the Ac-225 to the cancer cells whereby the Herceptin targets the HER-2/neu protein expressed on the cells while retention within said large liposomes of said radioactive decay intermediates produced by said radionuclide reduces the systemic release thereof.

In all aspects of this embodiment the cancer cells may comprise an ovarian carcinoma. The association of the Ac-225 within the small liposomal vesicles, the large liposomes and the molecules coating the outer membranes or incorporated into the outer membranes and the stabilizing agents incorporated into the aqueous phase are as disclosed supra.

In still another embodiment of the present invention there is provided an encapsulated alpha particle-emitting radionuclide comprising the alpha particle emitting radionuclide; small liposome vesicles entrapping the alpha particle emitting radionuclide; and a large liposome incorporating the small liposome vesicles where the large liposome has a diameter sufficient to retain a majority of the radioactive decay intermediates thereby encapsulating the alpha particle emitting radionuclide. The radionuclides and association of the radionuclide within the small liposomal vesicles and the biotin labeling thereof, the large liposomes and the molecules coating the outer membranes or incorporated into the outer membranes and the stabilizing agents incorporated into the aqueous phase, and the antibodies and target cells are as disclosed supra.

The present invention provides targeted delivery of alpha particle-emitting radionuclides and their alpha-emitting progeny as it relates, for example, to cancer therapy, such as for disseminated metastatic disease, using liposome-encapsulated alpha emitters. The systemic release of radioactive decay intermediates upon administration of an alpha particle-emitting radionuclide to an individual is reduced or even prevented by incorporating the radionuclide into the liposome either in the aqueous phase or in association with the membrane within large unilamellar liposomes (LUV) or within multivesicular liposomes.

The liposomes are coated with molecules that preferentially associate with a target cell such as anti-tumor antibodies, peptides, engineered molecules or fragments thereof. Encapsulation of Ac-225 within an immunoliposome reduces loss of radioactive decay intermediates from the targeting vehicle and, by extension, the target cell, such as, but not limited to, cancer cells, virally infected cells, autoimmune cells or inflammatory cells. The radioactive decay intermediates that remain sequestered within the multivesicular liposomes (MUVELs) are not systemically released into the individual.

The alpha particle emitting radionuclide may be incorporated into the aqueous phase as a chelation compound. Retention of radioactive decay intermediates may be facilitated by incorporating stabilizing agents, such as a phosphate buffer, insoluble metal binding polymer, resin beads, metal-binding molecules or halogen binding molecules, into the aqueous phase of the liposomes. Also, the pH of the liposome may be increased to facilitate retention. Additionally, the liposomes provided herein further comprise additional molecules to facilitate membrane fusion with target cells or to facilitate endocytosis by target cells.

The instant invention is especially useful for the delivery of the alpha particle-emitting radionuclides ²²⁵Ac, ²²³Ra, ²¹³Bi, and ²¹¹At. It is contemplated that beta emitting radionuclides that decay to alpha particle emitting daughters may be used. An example of such a beta emittor is ²¹²Pb which decays to ²¹²Bi, an alpha emittor.

To increase confinement of these intermediate daughters to the targeted tumor site, engineered liposomes of sufficient diameter containing actinium-225 are used. Ac-225 retention is sufficient within the aqueous phase of the liposome. However, the radionuclides localize to the membrane surface of the liposomes, thus causing considerable decrease of the daughter retention. Because of the recoil distance of the daughter nuclei following alpha particle emission, 650 to 1000 nm-diameter liposomes are required in order to achieve adequate (>50%), preferably (>60%), daughter retention while still allowing tumor cell irradiation by alpha particles.

Stable zwitterionic or cationic PEGylated phosphatidylcholine-cholesterol unilamellar liposomes of sufficient size can entrap multiple (>2) ²²⁵Ac atoms. Actinium-225 retention by zwitterionic liposomes was more than 88% over 30 days. Retention by cationic liposomes was lower at about 54% over the same time period. Retention in large liposomes, as measured by ²¹³Bi retention was lower, than smaller diameter. It is contemplated that this is due to a slower approach and localization of the actinium-chelate complex to the liposomal membrane.

However, even with a large unilamellar liposome (LUV) having sufficient diameter membrane localization of the radionuclides is still a key parameter in these liposome systems with concomitant loss of daughters. To retain greater than 60% of bismuth-213 activity within the liposome, the actinium must be entrapped within the aqueous phase and not associated with the membrane surface of the LUV. Encapsulating the actinium-225 within a small liposome which is then entrapped within the aqueous phase of the larger liposomal structure having a diameter of about 700 nm to about 800 nm forms multivesicular liposomes or MUVELs. This liposomal design allows for excess membrane within the larger liposomes and hence radionuclide localization within the core of the larger liposomes.

The instant invention is especially directed to the use of the liposomes for the delivery of alpha particle emitting radionuclides for the treatment of cancer. The relatively large size of liposomes, i.e., about 600-1000 nm in diameter, that is required for adequate bismuth retention is advantageous for therapy of disseminated cancers, such as ovarian cancer. However, use of the liposomes in this context is not restricted to ovarian cancer and may be efficacious in treatment of, inter alia, breast cancer with measurable liver or bone metastases. The instant invention also provides a method to reduce non-tumor specific uptake of the radionuclide containing liposomes into reticuloendothelial organs such as the spleen and liver by preinjecting empty liposomes into the individual to saturate absorption of liposomes into these organs.

To retain the intermediates at the tumor site, large, i.e., 600-1000 nm in diameter, engineered liposomes with encapsulated ²²⁵Ac and immunolabeled with anti-tumor antibodies, e.g., Herceptin antibody, are provided. Aside from the critical advantage of daughter retention, liposomal delivery of alpha-emitters also has the potential to increase the number of alpha-particles that are delivered per targeted cell. For example, depending on the lamellarity, an 800 nm multilamellar vesicle will have approximately 21×10⁶ lipids per vesicle (37). As described in the methods, ²²⁵Ac-loaded 800 nm vesicles are formed by co-incubation of 25×10¹⁷ cholesterol-phosphatidyl choline complexes with a given activity A₀ of ²²⁵Ac in a 2 ml volume. Extrusion of this yields approximately 1.2×10¹¹ vesicles, each containing at least 0.0334*A₀, with two in ten containing 2*0.0334*A₀, atoms of ²²⁵Ac when the encapsulation efficiency is 10%.

If, for example, 150 μCi are used, than each vesicle will contain a least 5, and two in ten will contain 10, atoms of ²²⁵Ac. Over a 30 to 40 day period each ²²⁵Ac atom will yield 4 alpha particle emissions. Assuming, conservatively, that only 10 liposomes are bound to each tumor cell, each cell would get irradiated by more than 10*5*4=200 alpha-particles. Again, assuming conservatively, that these decays occur on the surface, approximately ⅕ would traverse the nucleus for a cell where the nuclear diameter is 80% of the cellular diameter. The resulting 40 nuclear traversals would assure sterilization of targeted cells. A secondary advantage of liposomal targeting is the ability to simultaneously coat the liposomes with antibodies that target different antigens, thereby reducing the possibility of not targeting a particular population of tumor cells, e.g., ones that do not express a particular antigen.

Routes of administration of immunolabeled MUVELs are dependent on the disease targeted and are well-known to those of ordinary skill in the art. Because of the size of the liposomal structures required to contain the daughters, the approach is ideally suited for locoregional therapy, e.g. intraperitoneal, intrahepatic artery, intrathecal. However, the invention is not limited to such types of therapy. Additionally, such an artisan would be skilled in determining dosage and use of therapeutic radionuclides. As such, it is appreciated that the means and methods of use of unilamellar or multivesicular liposomes for targeted radionuclide delivery is not limited by the instant disclosure.

The following examples are given for the purpose of illustrating various embodiments of the invention and are not meant to limit the present invention in any fashion.

### EXAMPLE 1

Reagents

The lipids L-α-phosphatidylcholine from egg (EPC), 1,2-dipalmitoyl-sn-glycero-3-phosphoethanolamine-N-[methoxy(poly ethyleneglycol)-2000] (ammonium salt) (PEG-labeled lipid), 1,2-dipalmitoyl-sn-glycero-3-ethylphosphocholine (chloride salt), L-α-phosphatidylethanolamine-N-(4-nitrobenzo-2-oxa-1,3-diazole) from egg (NBD-PE), and L-α-phosphatidylethanolamine-N-(lissamine rhodamine B sulfonyl) from egg (purity>99%) (RhD-PE) were purchased from Avanti Polar Lipids (Alabaster, Ala.). Cholesterol, ascorbic acid, phosphate buffered saline (PBS), fluorexon (calcein) and Sephadex G-50 were purchased from Sigma (St. Louis, Mo.). DOTA (1,4,7,10-tetraazacyclododecane-1,4,7,10-tetraacetic acid) was obtained from Macrocyclics (Dallas, Tex.). Dithionite (sodium hydrosulfite, tech. ca. 85%) was obtained from Acros Organics (NJ). ²²⁵Ac was obtained from Oak Ridge National Laboratory (OakRidge, Tenn.).

### EXAMPLE 2

Liposome Preparation

Mixtures of phosphatidylcholine (EPC), cholesterol (1:1 molar ratio) and PEG-labeled lipids at 6 mole % of total lipid in CHCl₃ were dried in a rotary evaporator. For stability measurements, the lipids were resuspended in a 55 mM calcein solution in phosphate buffer, isosmolar to PBS, pH=7.4. For ²²⁵Ac passive entrapment, the lipids were resuspended in PBS containing chelated actinium complexes of ²²⁵Ac-DOTA to 50-100 μCi per ml (12) and DTPA. The lipid suspension was then annealled to 55° C. for 2 hours (24) (36). To make liposomes, the lipid suspension was then taken through twenty-one cycles of extrusion (LiposoFast, Avestin, Ontario, Canada) through two stacked polycarbonate filters having 100, 400 and 800 nm filter pore diameters. Unentrapped contents were removed by size exclusion chromatography (SEC) in a Sephadex G-50 (Aldrich, St. Louis, Mo.) packed 1×10 cm column eluted with an isotonic buffer. Ascorbic acid (8 mM) was coentrapped to minimize lipid oxidation due to radiation (39).

To make unilamellar liposomes for radionuclide binding measurements, the above procedure was repeated. The lipids were resuspended in a 138 mM sucrose solution at pH=7.4 isosmotic to PBS and extruded through filters with 100 nm pore diameter. Unentrapped sucrose was removed by dilution of the liposome suspension in PBS, followed by ultracentrifugation for 2 h at 142,000 g at 25° C. The pellet (10% v/v) was resuspended in PBS and used for the ²²⁵Ac binding measurements.

The unilamellar character of the zwitterionic and cationic liposomes was tested using dithionite. Dithionite ion S₂O₄— and the spontaneously produced .SO₂— radical react with the NBD-PE labeled lipids of the outer membrane layer and produce non-fluorescent derivatives (40). They diffuse very slowly through the bilayer, and thus allow the quantitative distinction of the inner and outer layer lipids. In our NBD-PE labeled vesicle preparations, unilamellarity was verified by a 51+4%, for zwitterionic liposomes, and 55±4%, for cationic liposomes, decrease of the initial fluorescence upon dithionite addition.

To make small liposomes for multilamellar vesicles the lipid suspension is sonicated in a bath sonicator for thirty minutes and extruded through two stacked 100 nm polycarbonate filters as above. Unentrapped contents were then removed by size exclusion chromatography (SEC), as above. The membranes of small liposomes can be labeled with biotin.

For coentrapment of smaller liposomes to make multivesicular liposomes, the lipids were resuspended in the solution of sonicated liposomes. The lipid suspension was then annealled to 55° C. for 2 hours. Larger liposomes whose outer membrane consists partially or entirely of the membrane of biotin-labeled small liposomes were removed by a streptavidin labeled Sepharose high-trap affinity column (HiTrap Streptavidin HP, Amersham Biosciences).

### EXAMPLE 3

Liposome Size Distribution Determination

Dynamic light scattering (DLS) of liposome suspensions was studied with an N4 Plus autocorrelator (Beckman-Coulter), equipped with a 632.8 nm He—Ne laser light source. Scattering was detected at 15.7°, 23.0°, 30.2°, and 62.6°. Particle size distributions at each angle were calculated from autocorrelation data analysis by CONTIN (41). The average liposome size was calculated to be the y-intercept at zero angle of the measured average particle size values vs sin²(θ) (42). All buffer solutions used were filtered with 0.22 μm filters just prior to liposome preparation. The collection times for the autocorrelation data were 1-4 minutes.

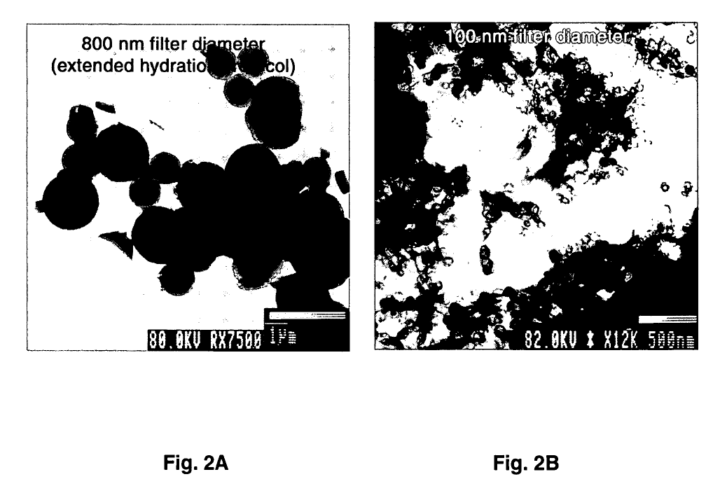

The measured average liposome sizes for the zwitterionic composition were 183±83 nm (100 nm filter diameter), 402±185 nm (400 nm filter diameter), and 646±288 nm (800 nm filter diameter). For the cationic composition the corresponding values were 184±74 nm (100 nm filter diameter), 415±153 nm (400 nm filter diameter), and 602±385 nm (800 nm filter diameter). Liposome size distributions were stable over a period of 30 days. Liposomes were also imaged by transmission electron microscopy using the negative staining method (FIGS. 2A-2B).

### EXAMPLE 4

Transmission Electron Microscopy (TEM)

To observe the size and external morphology of liposomes, a JEOL transmission electron microscope was used at 80 kV following the negative staining method. Liposome suspensions were added drop-wise to a 400-mesh copper grid coated with Formvar®. After allowing for liposome adhesion, excess sample was removed with filter paper. Staining was obtained with isotonic uranyl acetate solution (2%) in phosphate buffer.

### EXAMPLE 5

Liposome Stability Measurements

To study the liposome stability, i.e., the retention of entrapped contents, a fluorescent dye calcein was encapsulated at self-quenching concentrations. Different liposome sizes (100, 400 and 800 nm, filter pore diameter) were examined. The fluorescence intensity of liposome suspensions was measured by a fluorescence microplate reader (ex: 485 nm; em: 538 nm). Destabilization of the liposomal membrane causes calcein leakage from the liposomes. Calcein leakage is followed by dilution of calcein into the surrounding solution and relief of the fluorescence self-quenching effect, which results in increase of fluorescence intensity.

To normalize and properly compare different samples, Triton X-100 (4.5 wt %) was added into the suspension to achieve complete calcein release. To characterize liposome stability, the fluorescence self-quenching efficiency q of liposome suspensions was compared over the period of 30 days (q=Iₘₐₓ/I, where I is the measured fluorescence intensity before Triton X-100 addition, and Iₘₐₓ the maximum fluorescence intensity after Triton X-100 addition).

Release of calcein, due to liposome instability, results in dilution of the fluorophore into the surrounding buffer solution, relief of self-quenching and an increase of the fluorescence intensity. Fractional fluorescence self-quenching decrease due to calcein leakage from PEGylated liposomes over time is:

Δq_{day}_{_{—}}ₓ=(q_{day}₁−q_{day}_{_{—}}ₓ)/q_{day}_{_{—}}₁).

Both liposome formulations and all liposome sizes showed a small release of calcein within the first 24 hours post preparation. This may be caused by differences in osmolarity between the encapsulated calcein solution and the liposome surrounding solvent or by membrane defect relaxation. After the second day, and over the period of 30 days, all types of liposomes were stable. The effect of radiation on the stability of liposomes was studied by DLS. The liposome size distributions of samples that contained ²²⁵Ac did not change over time. The fractional fluorescence self-quenching decrease, Δq×100, is shown in Table 1 for zwitterionic and cationic liposomes over the period of 30 days. The uncertainties correspond to standard errors of repeated measurements.

TABLE 1
Percent decrease in self-quenching with time

| | Zwitterionic Liposomes | | | Cationic Liposomes | | |
|---|---|---|---|---|---|---|
| | Δq × 100 | | | Δq × 100 | | |
| Days | 800 nm | 400 nm | 100 nm | 800 nm | 400 nm | 1000 nm |
| 1 | 0 ± 2 | 0 ± 2 | 0 ± 3 | 0 ± 2 | 0 ± 2 | 0 ± 2 |
| 2 | 11 ± 2 | 14 ± 3 | 13 ± 3 | 16 ± 3 | 20 ± 2 | 20 ± 3 |
| 3 | 14 ± 2 | 14 ± 3 | 3 ± 3 | 14 ± 3 | 19 ± 3 | 17 ± 3 |
| 10 | 18 ± 2 | 18 ± 2 | 19 ± 3 | 17 ± 5 | 25 ± 2 | 24 ± 3 |
| 20 | 17 ± 2 | 16 ± 2 | 18 ± 3 | 20 ± 2 | 24 ± 2 | 26 ± 2 |
| 30 | 14 ± 3 | 15 ± 3 | 19 ± 6 | 19 ± 4 | 22 ± 4 | 27 ± 6 |

### EXAMPLE 6

Analysis of Indium-111 Leakage From Liposomes

To optimize the liposomal formulation and evaluate potential leakage of radionuclides from liposome constructs, indium-111 was used in place of Ac-225. Indium-111 (In-111) is easily detected by gamma counting and both indium-111 and actinium-225 form tri-chloride complexes. Thus, In-111 serves as a test model for liposomal encapsulation of Ac-225. The method described by Hwang et al. was used with minor modifications (43).

Liposomes were isolated by Sephadex™ chromatography and loaded with In-111 by incubation for 1 h at room temperature with a loading solution consisting of 6-10 μl 6.9 mM oxine in oxine sulfate in deionized water with 200 μl 1.8% NaCl/20 mM sodium acetate, pH 5.5. To this acetate buffer, an equal volume of ¹¹¹InCl₃ in 3 mM HCl was added to make the final loading solution. Loading was terminated by passage through an AGIX ion-exchange column. The fractions corresponding to liposomes were pooled and stored at 4° C. and 37° C. for evaluation of indium leakage.

At different times after ion-exchange chromatography, aliquots of the pooled liposomal fractions were drawn, chromatographed by size exclusion separation on a Sephadex™ column and counted for radioactivity in a gamma counter. The counts in liposomal fractions were expressed as a fraction of the radioactivity aliquoted from the original pooled sample. In selected samples, prior incubation with DTPA was included to ensure that retention of ¹¹¹In in liposomal fractions was not the result of leakage and equilibration of ¹¹¹In between extra- and intra-liposomal ¹¹¹In in the pooled sample.

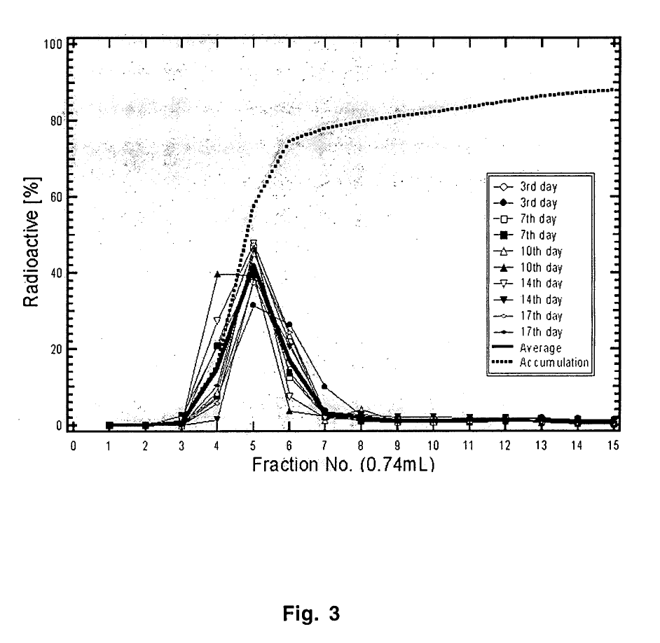

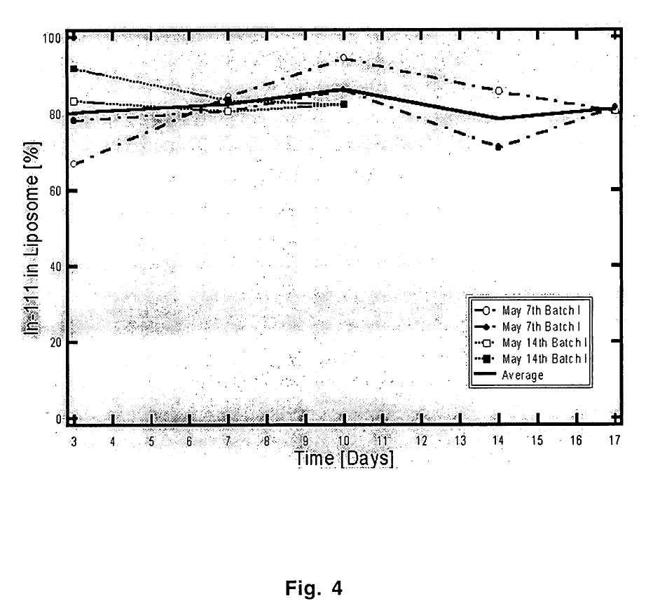

Following ion-exchange chromatography, approximately 20% of the radioactivity remains on the ion-exchange resin, and close to 80% in the liposomal fractions, yielding an encapsulation efficiency of approximately 80%. The radioactivity collected in each fraction after Sephadex™ column chromatography at different times after ¹¹¹In encapsulation is depicted in FIG. 3. The results show a generally time-invariant profile following size-exclusion separation. FIG. 4 depicts the retention of ¹¹¹In in liposomes as a function of time after loading. The fraction of ¹¹¹In retained within liposomes appears to remain constant at approximately 80% over a prolonged time-period, indicating that retention of Ac-225 within liposomes is possible.

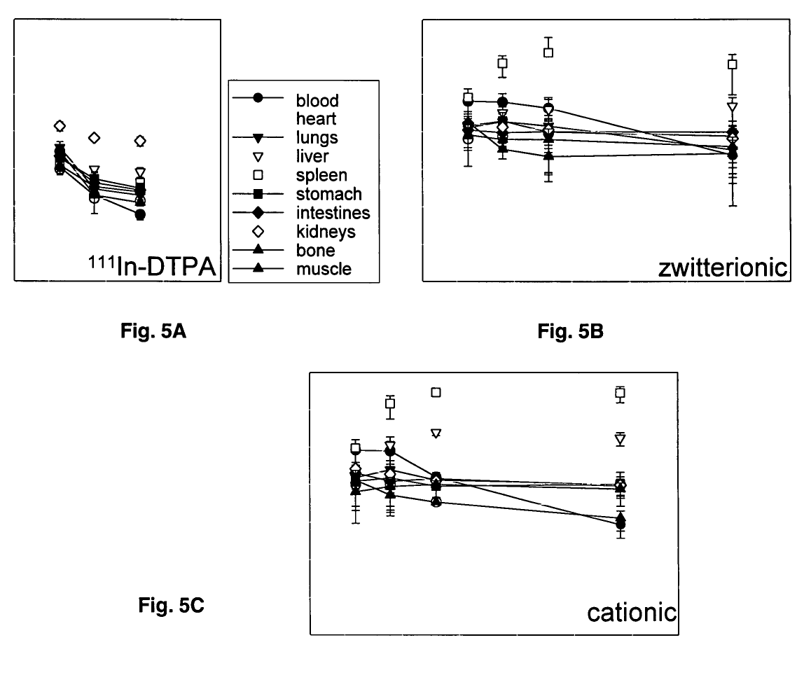

The stability of zwitterionic and cationic large liposomes was tested in vivo (FIG. 5A-5B). Liposomes are loaded with high concentrations of calcein and administered intraperitoneally. After 6 hrs the liposomes' fluorescence is minimal indicating the liposomes are intact. After addition of Triton-x 100, the liposomal membrane is dissolved and calcein is released resulting in an increase in fluorescent signal. This demonstrates that liposomes will remain intact in the peritoneum which is a requirement for actinium-225 delivery. Release of the actinium-225 into the peritoneum results in actinium-225 in the blood and kidney toxicity.

### EXAMPLE 7

Determination of Loss Rate of Daughters From Ac-225 Ecapsulated Liposomes Using Indium-111 Model

Gamma counting provides information regarding the presence of daughter radionuclides within the liposomes. Liposomes were incubated for 1 hr with Ac-225. To determine loss rate of daughter radionuclides from liposome-encapsulated Ac-225 activity, Ac-225 containing liposomes were separated from free Ac-225 by elution through a Sephadex™ column. The fractions corresponding to the liposomes, i.e., fractions 4-6, based on In-111 encapsulation, were pooled and counted on a gamma counter using windows appropriate for detection of the Fr-221 and Bi-213 photopeaks, 218 and 440 keV, respectively. A one minute counting interval was selected and counting was performed overnight. Fr-221 counts were decay corrected to the start of the one minute counting period. At the end of separation and 10 to 20 h later, if possible, the column also was counted in a dose calibrator.

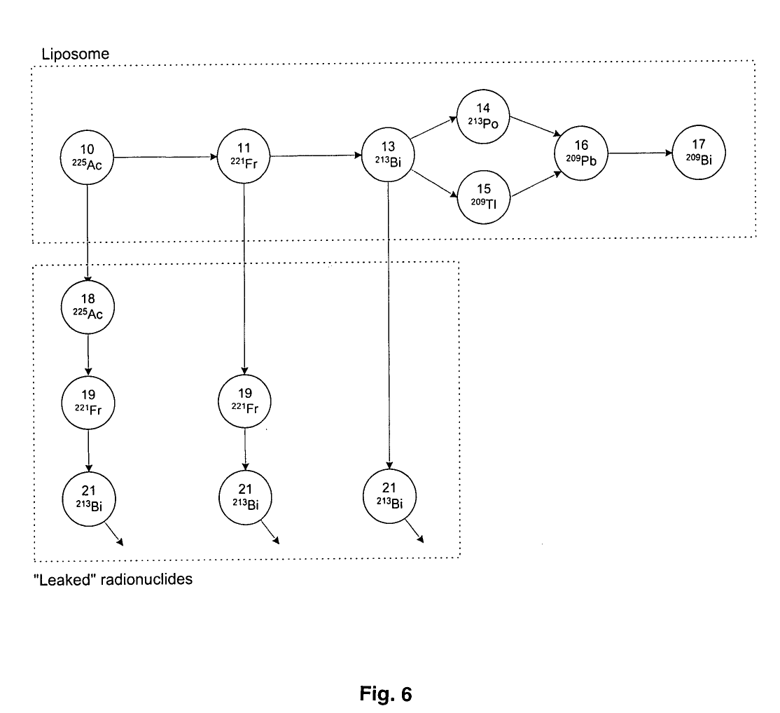

The results were analyzed using the model of Ac-225 radioactive decay shown in FIG. 6. Sub-compartments 10-17 correspond to decays that occur within liposomes. Loss of daughter or parent radionuclide is depicted by a “leak” rate from the liposome compartments to corresponding sub-compartments in the “leaked” radionuclides compartment. The transfer rate from each sub-compartment within liposomes to the extraliposomal compartment reflects a rate of loss from liposomes. This rate may be described in terms of a loss or clearance half-life, the amount of time required for Ω of a particular radionuclide to diffuse out of the liposome. Transfer rates within a compartment correspond to physical decay, whereas those that cross between the two compartments correspond to transfer of radionuclide from one to the other compartments. Since astatine-217, which has a 32 msec half-life is not resolved by this model, it is lumped with Fr-221.

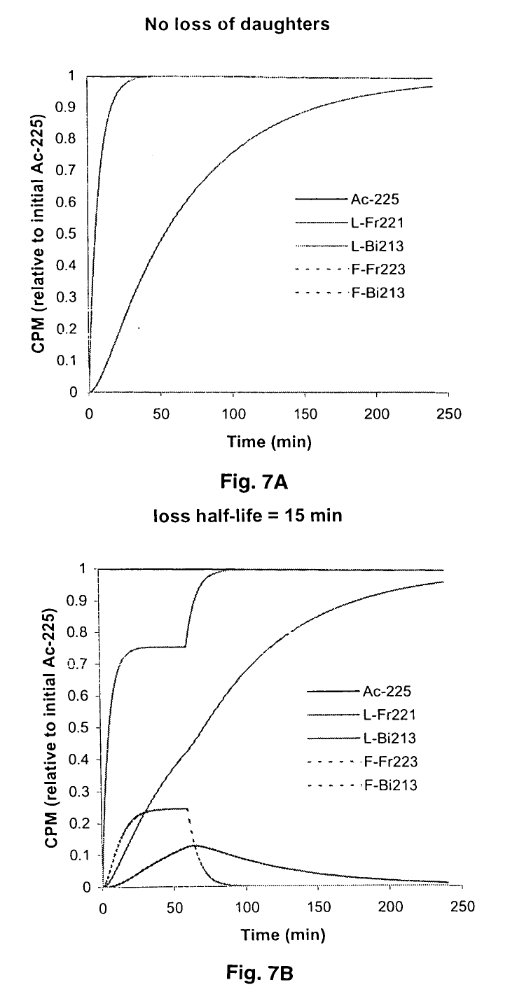

A sample simulation using this model is depicted in FIGS. 7A and 7B. FIG. 7A shows the results of a simulation in which complete retention of all radionuclides was assumed. Results are expressed relative to the initial activity of Ac-225 encapsulated by the liposomes. The first solid line shows the decay of Ac-225 while the other solid curves show the rise in daughter radioactivity within liposomes as equilibrium between the parent and daughters is reached. Note that because of its short, five minute half-life, Fr-221 achieved equilibrium much more rapidly than Bi-213, which has a 45.6 min half-life. The two dotted lines, corresponding to Fr-221 or Bi-213 activity not within liposomes, are just visible at zero throughout the simulation duration.

FIG. 7B shows a simulation assuming loss of Fr-221 at a rate of 0.046 min⁻¹ which is equivalent to a loss half-life of 15 min. All other loss rates were set to zero. This means that Bi-213 generated within liposomes was assumed to remain there. Complete retention of Ac-225 was also assumed in this simulation.

As shown by the rise in both liposome-associated and free daughters (solid and dashed lines, respectively), equilibration and loss of daughters was allowed to occur during the 1-hr incubation period. After separation, and during counting of liposomal fractions, the distinction between free and liposome-encapsulated radioactivity was no longer possible. Therefore, the loss rate is turned off so that the solid curves correspond to total daughter radioactivity associated with the liposomal fractions, including daughters that were leaking out. The dashed curves reflect the physical decay of daughter radionuclides remaining in the column or in the liposome-free fractions. Although the latter could be assayed for radioactivity of Fr-221 and Bi-213, the method for assessing daughter loss rates was based upon counting the liposomal fractions.

Analysis of Daughter Loss From Unilamellar Liposomes Using Different Loss Rates

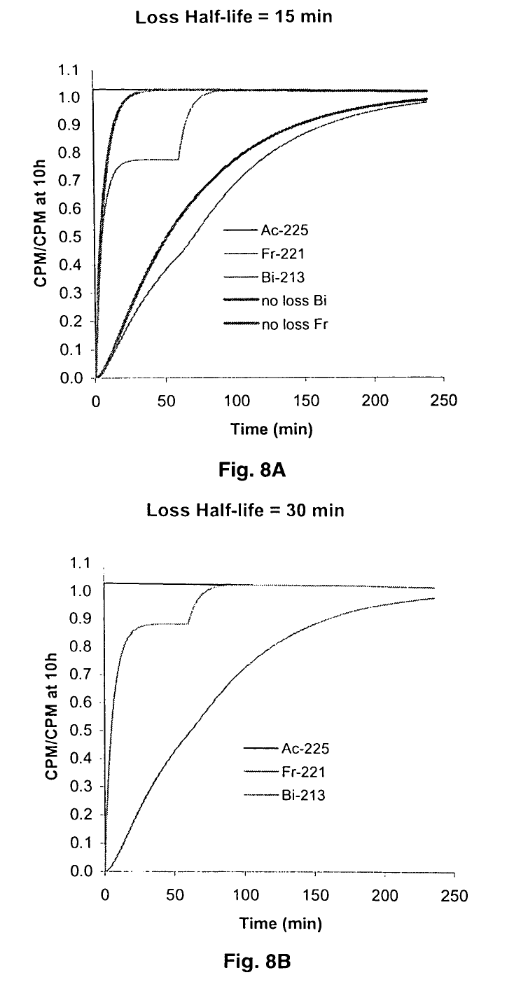

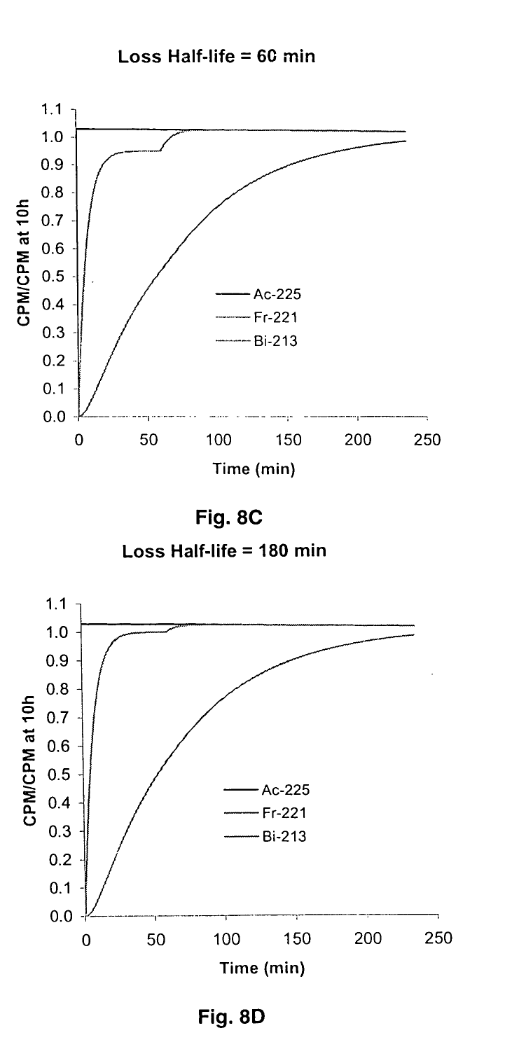

FIGS. 8A-8D depict simulations obtained using four different loss rates. The curves associated with counting of liposomal fractions are shown without the corresponding curves for free daughters collected on the column or in the liposome-free fractions. All other conditions are as described above. The CPM values are normalized by dividing by CPM expected after 10 h (i.e., at equilibrium).

In FIG. 8A where the loss half-life is modeled at 15 min the curves corresponding to a simulation that did not include loss of Fr are shown for comparison with the simulation that included loss. The level of Fr-221 and Bi-213 activity immediately after separation was sensitive to the loss half-life of Fr-221. This is clearly evident for Fr-221 since the loss rate impacted the equilibrium level (plateau) that was reached. It was less evident for Bi-213 since it does not reach equilibrium in 1 h. Once the separation had occurred, i.e., after 60 minutes, the distinction between free and liposome-associated daughter activity was lost over time as the daughters reached equilibrium with the parent. Correspondingly, as the measurement of daughter activity was delayed relative to the time of separation, the ability to distinguish the different loss rates is also reduced.

Relationship Between Loss Rate and Daughter Activity

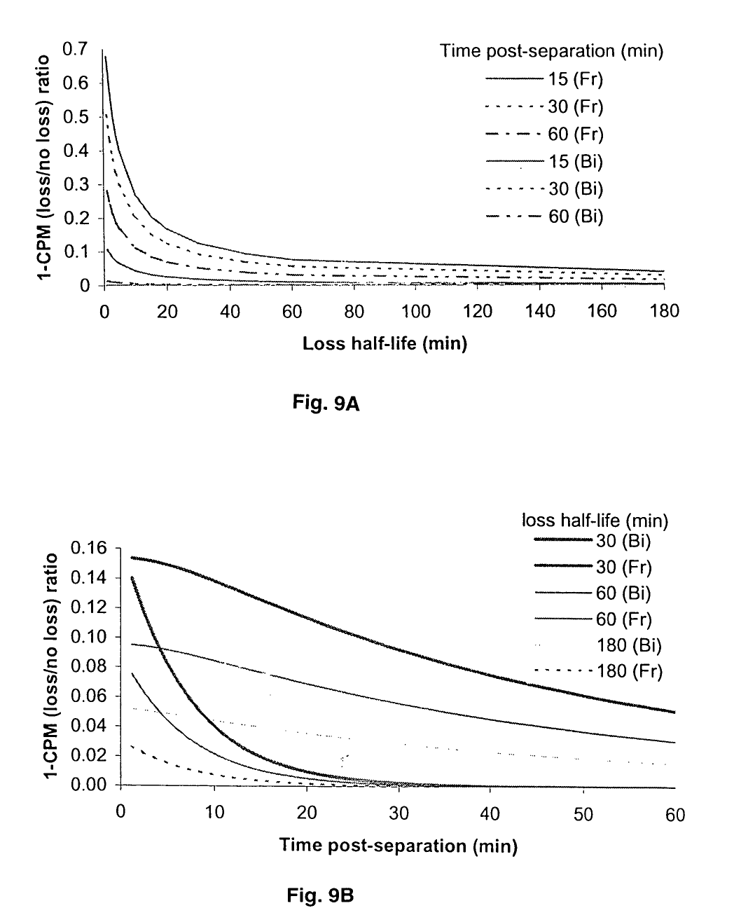

The relationship between loss rate and the levels of daughter activity at different measurement times after liposome separation is shown more directly in FIGS. 9A-9B. FIG. 9A shows the normalized count rate ratio, as presented in FIGS. 8A-8D, of each daughter divided by the corresponding count rate ratio assuming no loss of Fr-221 and then subtracted from one to yield curves that approach zero over time. Results are plotted against different loss half-lives and for measurement times of 15, 30 and 60 minutes after separation. If counting is started immediately after separation and carried out overnight, these data are available.

FIG. 9B is a different representation of the data used to generate FIG. 9A. Loss rate sensitivity is plotted against the time post-separation at which the liposomal fractions are counted. Curves are provided for three different loss half-lives: 30, 60 and 180 min.

Determination of the Loss Rate that Yields a Negligible Absorbed Dose to Kidney From Bi-213

To determine the loss rate that yields a negligible absorbed dose to kidneys from Bi-213, a 5 day biological half-life of the Ac-225 construct is assumed and a 25 d simulation is plotted. Subsequently, the Bi-213 released for different Fr-221 release rates is integrated and used to perform rough estimate of dosimetry. 25 d=36000 min=simulation time 5 d half-life=9.627e⁻⁵

### EXAMPLE 8

Biodistribution of Zwitterionic and Cationic Large Liposomes

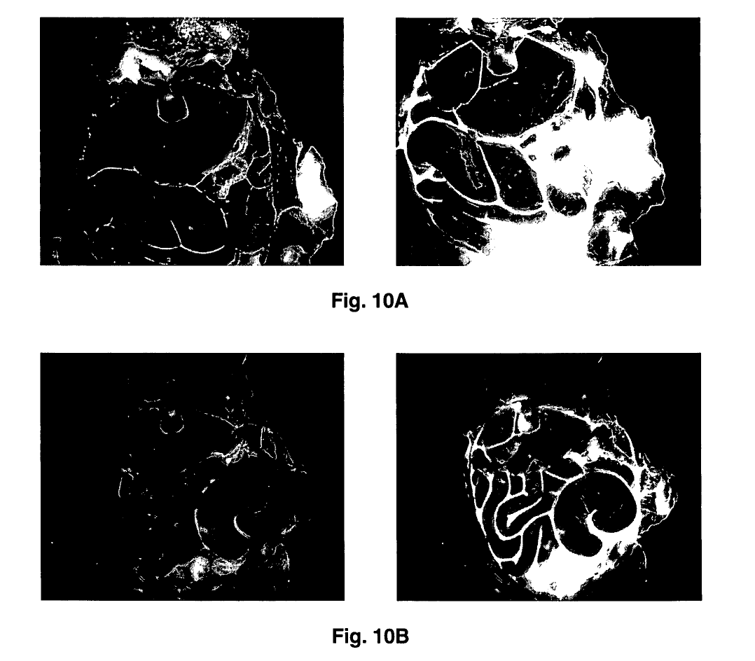

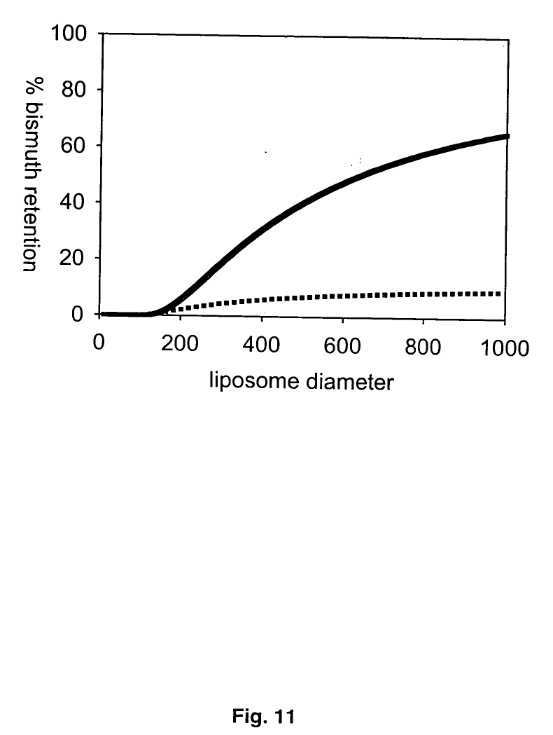

As shown in FIG. 10A, injected DTPA-chelated In-111 will localize to the kidneys more than to other tissues. Zwitterionic (FIG. 10B) and cationic liposomes (FIG. 10C) containing DTPA-chelated indium-111 localize predominantly to the spleen and to the liver. Over time the percentage per gram of tissue tested of the original amount of In-111 found in the spleen and liver increases while that in blood and other tissues such as heart, lungs, stomach, intestines, kidneys, bone, and muscle remain constant or decrease. Localization by cationic liposomes is slightly greater than that by zwitterionic liposomes. Preinjection with empty large liposomes to saturate the reticuloendothelial organs will reduce non-specific radionuclide uptake by the spleen and liver.

### EXAMPLE 9

Theoretical Model of Daughter Retention

To evaluate the theoretical limits of daughter entrapment, a model was developed using nuclear theory and geometry. The nuclear recoil distances of the alpha decays of the actinium daughters are not well established in aqueous media, but estimates to within 10-20% were made (44), using a standard computer model (SRIM, Stopping and Range of Ions in Matter, James Ziegler, http://www.srim.org/) (45). The range of the recoil distances for francium, astatine and bismuth were estimated as 81.7, 86.5 and 94.7 nm respectively. Since these differ by less than the experimental uncertainties, both in the recoil range estimates and in our experimental measurements, a single recoil distance of 87.6 nm was used to simplify further calculations.

Using this model, unilamellar liposomes were considered. Each entrapped radionuclide was distributed uniformly within the aqueous volume of a liposome. Thus, each disintegration has a fixed probability f for daughter ejection. The probability of ejection f_{r,rd} for a disintegration, which occurs a distance r from the center of a liposome of radius rₗ may then be calculated from geometry whre r_{d} is the recoil distance. The probability is calculated by averaging the ejection probability f_{r,rd} over the possible locations of the radionuclide, r:

$${f}_{r , {r}_{d}} = \begin{matrix}1 - \frac{2 {rr}_{d} - {r}_{l}^{2} + {r}^{2} + {r}_{d}^{2}}{4 {rr}_{d}} , r > {r}_{l} - {r}_{d} \\ 1 , r < {r}_{l} - {r}_{d}\end{matrix}$$

$$f = {}_{0}^{{r}_{l}} 4 π {f}_{r , {r}_{d}} {r}^{2} dr / {}_{3}^{4} π {r}_{l}^{3}$$

Retention of bismuth requires three successive decays without escape, so the probability is f³.

### EXAMPLE 10

Theoretical Determination of Liposome Size for Daughter Retention

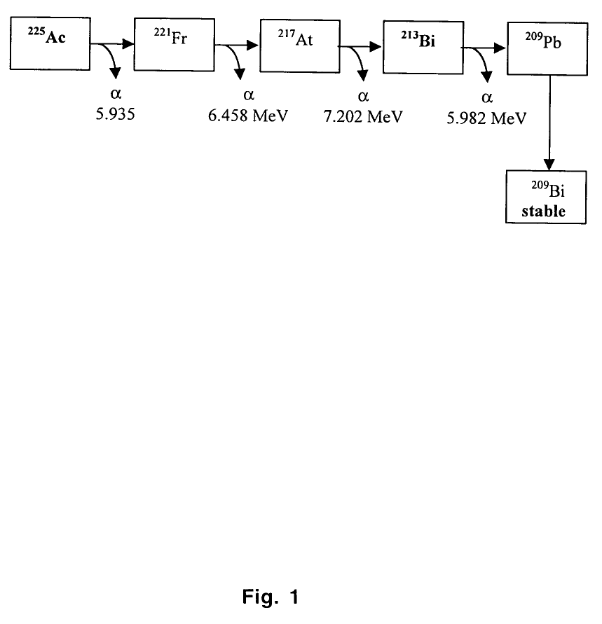

The mechanism for daughter loss from the liposome interior is likely to be nuclear recoil. The theoretical fraction of ²¹³Bi retention as a function of liposome size is shown in FIG. 1 assuming a recoil distance of 87.6 nm: average recoil distance in water calculated using SRIM. Bismuth-213 is the last alpha-emitting intermediate on the ²²⁵Ac scheme and thus retention of ²¹³Bi requires retention of both ²²¹Fr and ²¹⁷At. Adequate ²¹³Bi retention, (>50%), requires large liposomes (>650 nm in diameter).

Theoretical retention was also calculated assuming that ²²⁵Ac and its daughters localize at the inner liposome membrane; this represents a worst-case scenario. In this case, the maximum ²¹³Bi retention approaches 1/2³=12.5% for large liposomes. The retention is 8.1% for 650 nm-diameter liposomes. Half the recoils from the surface of an infinitely large vesicle would result in daughter ejection. Thus, if even a fraction of the daughters of ²²⁵Ac (or of ²²⁵Ac itself) associate with the liposomal membrane, the ²¹³Bi retention will be significantly reduced.

Actinium and Bismuth Retention Measurements

To experimentally test the retention of ²²⁵Ac and its daughters by the liposomes, the γ emissions of ²²¹Fr and ²¹³Bi were measured using a Packard Cobra Gamma Counter (Packard Instrument Co., Inc., Meriden, Conn.). Under steady state conditions, i.e., after 24 hours, the decay rate of each species in the decay chain must be equal. Thus, at steady state, the decay rate of either francium or bismuth can be used to determine the actinium concentration. Measurements of the kinetics of ²¹³Bi activity (t_{1/2} is 45.6 min) in liposome fractions separated from the parent liposome population and the unentrapped radionuclides, at different times, allow for estimation of the stability of ²¹³Bi entrapment. For each liposome population, measurements of ²¹³Bi activity were made following rechromatography (SEC, Sephadex G-50) at various time points.

Retention of ²²⁵Ac by Liposomes

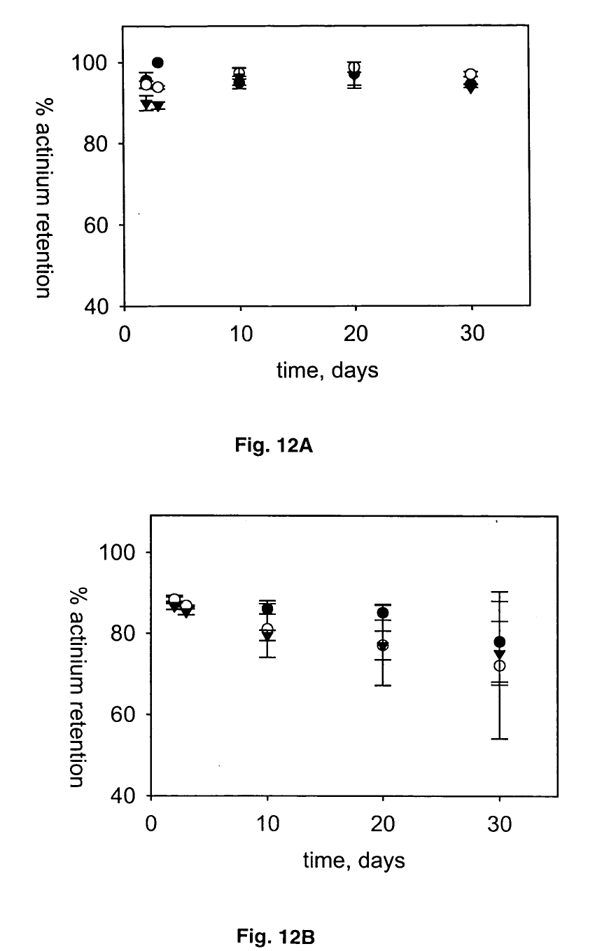

To entrap ²²⁵Ac in liposomes, chelated actinium complexes (225Ac-DOTA) were included in the phospholipid suspensions. Liposomes were extruded to different sizes, and unentrapped ²²⁵Ac was separated by size exclusion chromatography (SEC). As stated previously, ²²⁵Ac retention in liposomes can be determined by the ²²¹Fr or ²¹³Bi activities at steady state. At different time points, liposome fractions were separated from free ²²⁵Ac by SEC, and the gamma photons of ²¹³Bi decay at steady-state were used to determine ²²⁵Ac retention. Independent of time, the ²²⁵Ac activity retained in the zwitterionic liposome fractions was >88% (FIG. 120A). Actinium-225 retention decreased (FIG. 12B) over time in the cationic liposomes (cationic lipid: 10 mole % of total lipid), but even after 30 days the retention was more than 54%. Maximum ²²⁵Ac encapsulation efficiency in liposomes, using passive entrapment, was 10%.

Retention of ²¹³Bi by Liposomes

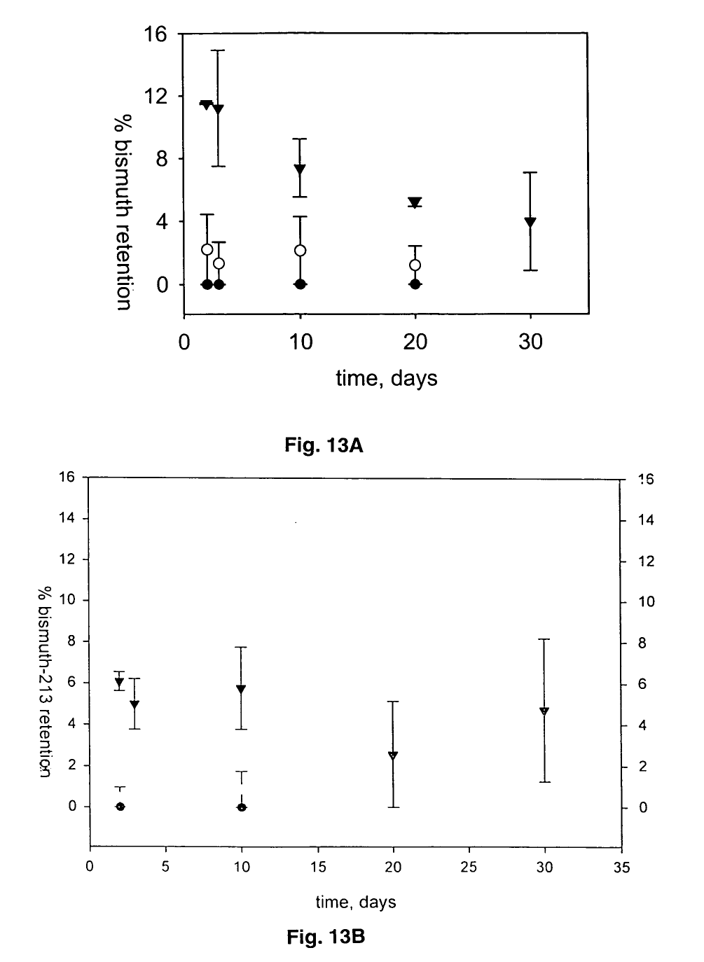

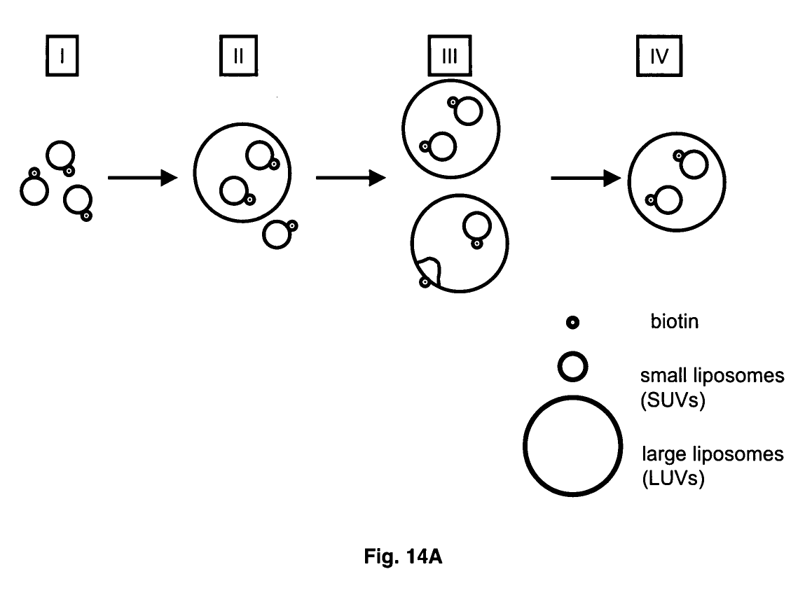

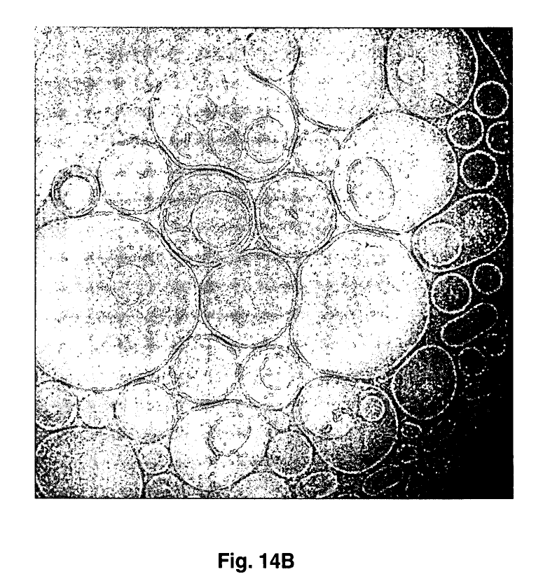

The retention of 21³Bi by liposomes was studied as a function of liposome size and composition. For each liposome population, measurements of ²¹³Bi activity were made following rechromatography at various time points (FIGS. 13A and 13B). For large liposomes, retention was much less than theoretically predicted. For both liposome compositions and all sizes examined, the values of ²¹³Bi retention were consistent with the theoretical results for localization of the radionuclides on the liposome membrane. The initial drop in FIG. 13A for zwitterionic 800 nm liposomes (V) may be explained by slower approach and localization of the actinium-chelate complex to the liposomal membrane.

### EXAMPLE 11

Binding Measurements of ²²⁵Ac and ²²⁵Ac-DOTA to Liposomes

Different activities of ²²⁵Ac and ²²⁵Ac-DOTA were mixed in PBS with sucrose-loaded liposomes and the system was allowed to equilibrate for at least one hour at room temperature. Rhodamine-labeled zwitterionic and cationic liposomes were present at the same concentrations as those used for the measurement of ²²⁵Ac and ²¹³Bi retention by the above liposomes. After ultracentrifugation for 2 h at 142,000 g, (25° C.), the supernatant (the top 90% of the sample volume) was promptly removed. Lipid in both supernatant and pellet was measured using the rhodamine fluorescence intensity (ex: 544 nm, em: 590 nm). Actinium-225 activity concentration was measured by the gamma emissions of ²¹³Bi decay at equilibrium.

The calculated amount of membrane bound ²²⁵Ac was corrected for the amount of lipid that remained in the supernatant, as follows: β, the fraction of bound ²²⁵Ac, was defined as the ratio of the bound ²²⁵Ac to the total ²²⁵Ac in the sample. It was assumed that the concentration of unbound ²²⁵Ac in the pellet [Ac]_{p,f} and in the supernatant were equal, since the distribution of free ²²⁵Ac atoms along the tube axis should not be influenced by the separation process. Thus, for the pellet, the average amount of ²²⁵Ac bound per lipid (B), would be: B=([Ac]_{p,t}−[Ac]_{p,f})/[L]ₚ, where [Ac]_{p,t}, is the total concentration of ²²⁵Ac in the pellet and [L]ₚ is the lipid concentration in the pellet. Since not all the liposomes were found in the pellet, B was used to calculate the bound ²²⁵Ac in the supernatant, and the true free ²²⁵Ac concentration.

The liposome concentration was kept constant and equal to that used when determining retention of ²²⁵Ac and ²¹³Bi and the radionuclide activity was varied over four orders of magnitude, encompassing the activities used in the determining retention. Significant membrane binding of ²²⁵Ac was observed with zwitterionic and cationic liposomes. The number of radionuclide atoms was low compared to the number of binding sites present determined by the concentration of lipid molecules, thus, no saturation on the binding profile was observed. The partition constants Kₚ (Kₚ=C_{bound} C⁻¹_{free} L⁻¹, C, L [=] M) were expressed as the ratio of bound to free radionuclide atoms, normalized to the concentration of lipid present (L). For the zwitterionic membranes Kₚ's were 420 (for ²²⁵Ac) and 60 (for ²²⁵Ac-DOTA), and for the cationic membranes: 550 and 50, respectively. In our measurements, 30-35% of ²²⁵Ac was bound to membrane under the experimental conditions (accessible lipid L=1 mM).

### EXAMPLE 12

Multivesicular Liposomes (MUVELs) for Enhanced Retention of Actinium-225 and its Daughters

Multivesicular liposomes were prepared as described for small liposomes containing self-quenching amounts of calcein. MUVELs were separated from unentrapped small liposomes by size exclusion chromatography. In addition, during the MUVEL preparation at the second step of annealing, various liposomal structures may be formed whose external membrane originates from small liposomes, i.e., unentrapped fused small liposomes that formed larger structures or small liposomes fused with large liposomes.

These structural forms, independently of their size, should be removed from the MUVEL suspension. Radionuclides were shown to localize on the membrane surface, and they will bind to the membrane of small liposomes. Thus, any part of these membranes should be removed from the surface of the final large liposomal structures, otherwise, the daughter retention will decrease dramatically.

To separate these forms from MUVEL, the membrane of small liposomes was labeled with biotin. Use of streptavidin coated affinity column removes all of the above structures (FIG. 14A). It is contemplated that to minimize these fusion events, different liposome compositions can be used for the sonicated small liposomes which have transition temperatures significantly higher than 55° C. that is used in the preparation protocol. Further structural characterization may be examined by cryo-TEM (FIG. 14B). This method allows for direct tomographic imaging of the lamellae at resolution higher than 100 nm.

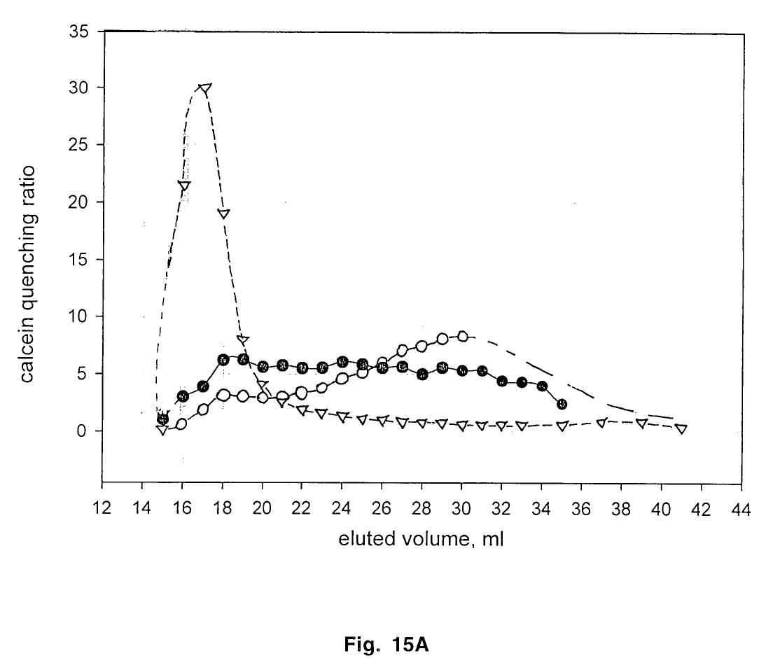

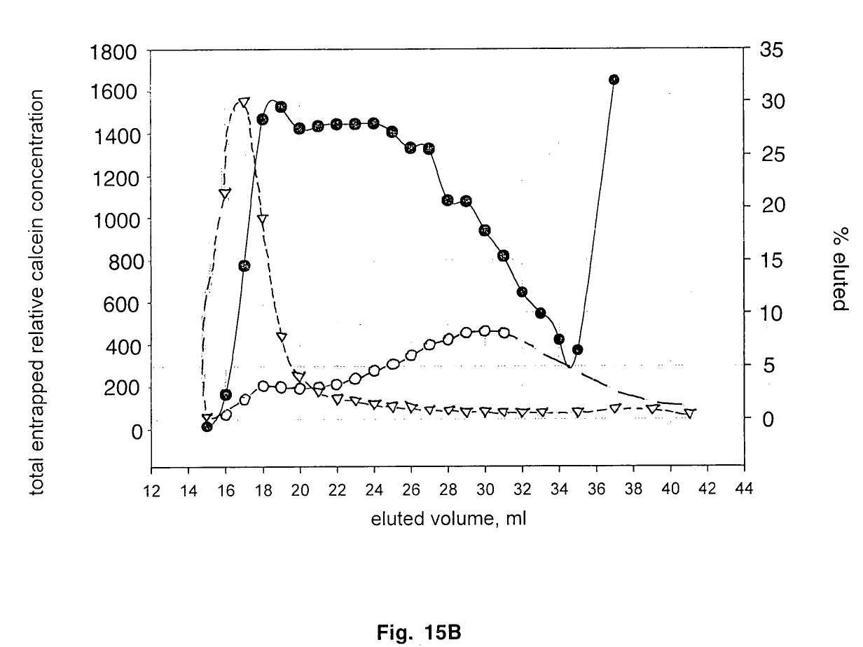

The fluorescence intensity of each fraction was measured before and after triton X-100 addition; the ratio of the fluorescence signals is shown in FIG. 15A. Quenching of calcein in the fractions 16-17-18 where the large liposomes mainly elute implies the presence of entrapped small liposomes in the large 800 nm liposomes. In addition, as shown in FIG. 15B, the total concentration of calcein after triton x-100 addition per MUVEL fractions 16-18 is indicative of the amount of small liposomes entrapped.

Stability of MUVELs

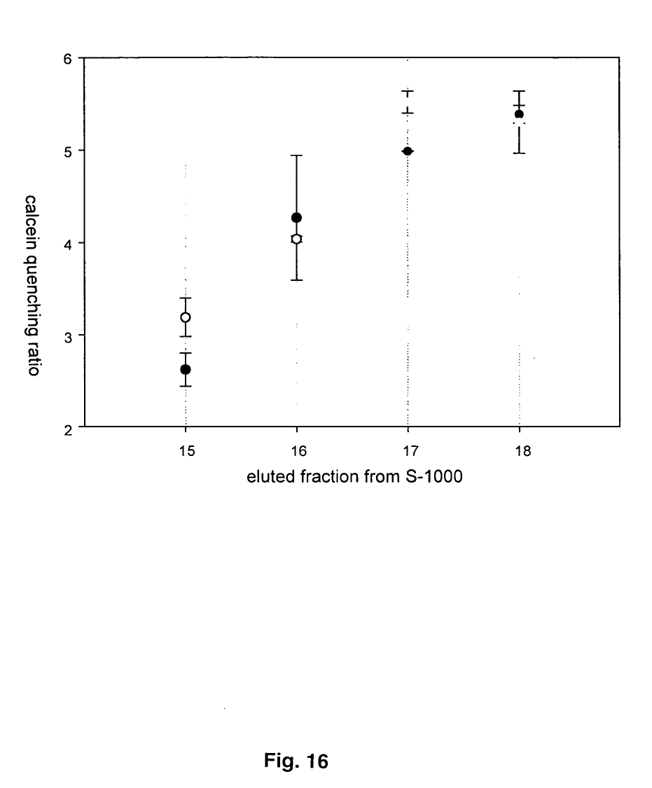

To study the stability of MUVELs, the quenching ratio of each fraction was followed in time (FIG. 16). After four days (closed symbols) the relative quenching ratios of the fractions shown are not significantly different. The calcein quenching ratio of small liposomes that have not been encapsulated into the MUVELs was stable during the above time period and of higher value: 9.6±1.2, indicating that calcein leakage has occurred in the encapsulated small liposomes.

### EXAMPLE 13

Retention of Actinium-225 and Bismuth-213 in MUVELs

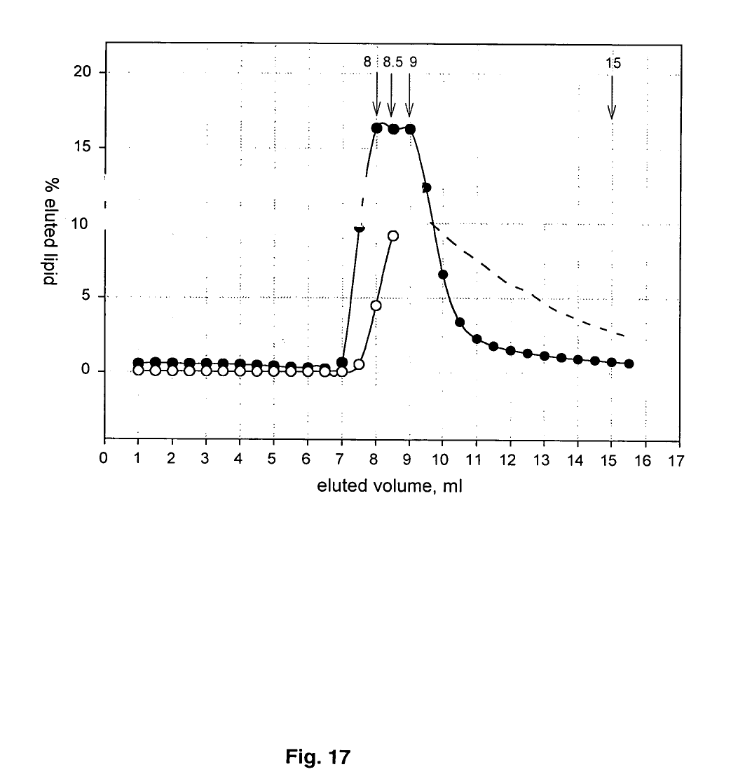

Small sonicated liposomes were prepared as described. Actinium was passively entrapped in small liposomes and unentrapped actinium was separated using size-exclusion chromatography (Sephadex G-50). Small liposomes were then entrapped in large liposomes extruded through an 800 nm diameter filter. MUVELs were separated by S-1000 (FIG. 17) and each fraction was purified by streptavidin labeled affinity column. Radionuclide retention results are shown for the fractions indicated with arrows (8, 8.5, 9, 15). Retention was determined as for unilamellar liposomes. For each vesicle population, measurements of ²¹³Bi activity were made following rechromatography at various time points.

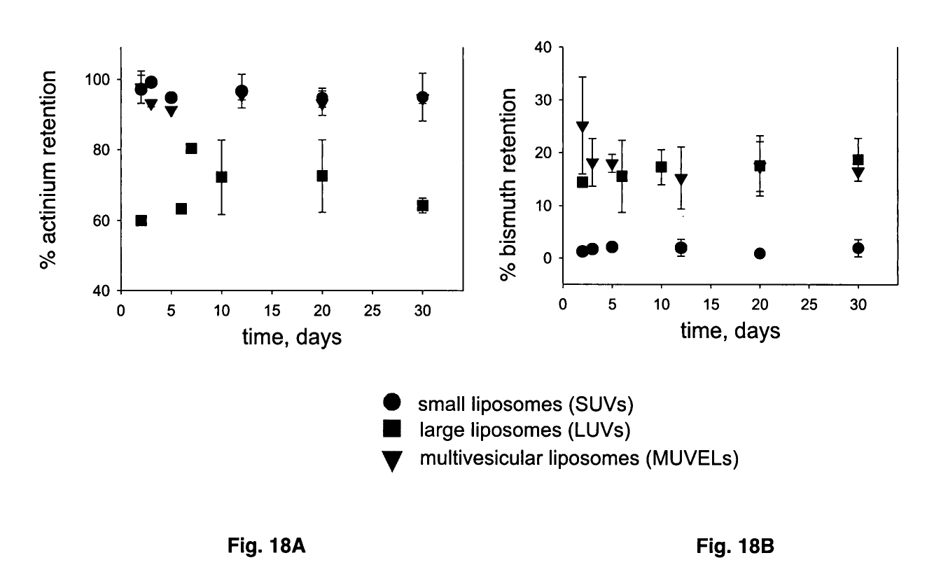

In FIG. 18A the retention of actinium-225 was then measured for small liposomes (SUV), large liposomes (LUV) and multivesicular liposomes (MUVEL) over the period of 30 days. SUVs and MUVELs demonstrate good actinium retention with MUVELs yielding about 98% Ac-225 retention. LUVs have a lower actinium retention as the actinium can localize to the membrane surface. As shown in FIG. 18B bismuth-213 retention is significantly improved in MUVELs and LUVs at about 20%. Unentrapped SUVs do not retain bismuth. The decrease with time of bismuth retention may possibly be explained by leakage of actinium from the entrapped small liposomes as in FIG. 18A, into the encapsulated aqueous compartment of the large liposomes. This would result in localization of the escaped radionuclides on the membrane surface of the large liposomes and thus decrease of bismuth retention.

As Bi-213 is the last daughter in the Ac-225 decay series, retention of even 20% of Bi-213 means that the retention of all the daughters improves delivery of alpha particles to a tumor about 260% over that of Ac-225 alone. This reduces the amount of radionuclide administered for effective therapy with concomitant reduction in toxicity.

### EXAMPLE 14

Targeted Delivery of Immunolabeled MUVELs

Preparation of Immunoliposomes

Antibodies are attached at the terminal end of the PEG chains of the vesicles; this geometry provides exposed antibody molecules that protrude from the vesicle for unhindered antigen recognition. In particular, vesicles are prepared with 1-3% mole carboxy-terminated PEG-lipids. The carboxy terminus is modified to an amine reactive NHS (N-Hydroxysuccinimide) ester by mixing the NHS and the dehydrating agent EDC (1-ethyl-3-(3-dimethylaminopropyl)carbodiimide hydrochloride) in the vesicle suspension. The vesicles are immunolabeled by reaction of the modified carboxy-termini with the epsilon amine groups of lysines on antibodies (46). Immunoliposomes are then separated from the unreacted antibodies by a Sepharose 4B size exclusion column preequilibrated with PBS (47).

Murine Model

In more than 40 mouse experiments, intraperitoneal injection of 5×10⁶ previously passaged SKOV3 cells in 4-6 week old female Balb/c nu/nu mice has yielded 100% tumor take. By three weeks, discrete, mm-sized tumor nodules are observed around the spleen; in many cases such nodules are also found on the surface of the GI tract and liver. By four weeks discrete tumor nodules are replaced by contiguous masses of tumor around the spleen, GI tract and liver. At this time ascites fluid is also observed.

Biodistribution of Herceptin-Labeled Immunoliposomes

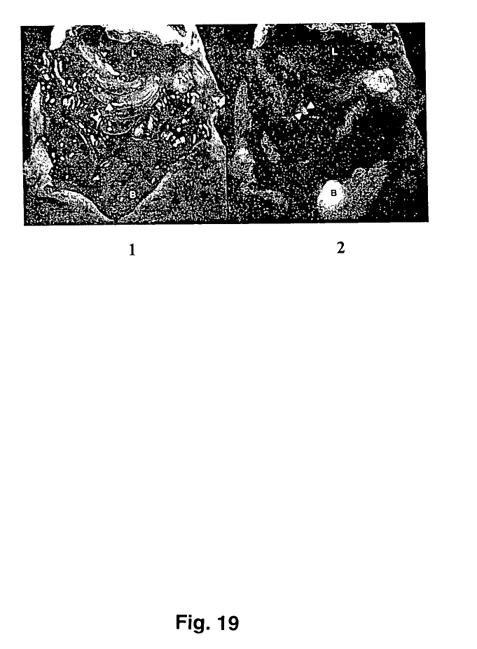

MicroPET and MRI-based pre-clinical biodistribution and localization data have been obtained using ⁸⁶Y-Herceptin (HER) antibody (anti-HER2/neu) against ovarian carcinoma (38-39). Radiolabeled Herceptin Ab was shown to localize to sites of disease with minimal normal organ uptake. Optical images of FITC-Herceptin have also been obtained to provide a high resolution assessment of Herceptin targeting as shown in FIG. 19. This methodology is also used to evaluate tumor targeting of liposomes immunolabeled with Herceptin.

Efficacy of Immunoliposome Against IP-Disseminated Ovarian Carcinoma

To evaluate the fate of immunoliposomes, e.g., Herceptin-labeled immunoliposomes, in vivo, both the aqueous and the lipid membrane compartments are labeled with fluorescent markers. The aqueous compartment, i.e., contents, is labeled by entrapping calcein as described for MUVELs. The membrane is labeled with NBD and rhodamine-tagged lipids to allow for fluorescence energy transfer. Thus, both the entrapped calcein and rhodamine are excited with the same laser light source. This enables simultaneous and independent detection of both compartments using different filters, making it possible to detect endocytosis/fusion of vesicles as well as vesicle integrity by multi-color fluorescence imaging and microsopy.

The following references were cited herein:

1. Jurcic J, McDevitt M, Sgouros G, Ballangrud A, Finn R, et al. Targeted alpha-particle therapy for myeloid leukemias: A phase I trial of bismuth-213-HuM195 (anti-CD33). Blood; 90:2245 (1997).

2. Sgouros G, Ballangrud A, Jurcic J, McDevitt M, Humm J, et al. Pharmacokinetics and dosimetry of an alpha-particle emitter labeled antibody: 213Bi-HuM195 (anti-CD33) in patients with leukemia. J Nucl Med; 40:1935-46 (1999).

3. Zalutsky M, Cokgor I, Akabani G, Friedman H, Coleman R, et al. Phase I trial of alpha-particle-emitting astatine-211 labeled chimeric anti-tenascin antibody in recurrent malignant glioma patients. Proceedings of The American Association for Cancer Research; 41:544 (2000).

4. Behr T, Behe M, Stabin M, Wehrmann E, Apostolidis C, et al. High-linear energy transfer (LET) alpha versus low-LET beta emitters in radioimmunotherapy of solid tumors: therapeutic efficacy and dose-limiting toxicity of 213Bi-versus 90Y-labeled CO17-1A Fab′ fragments in a human colonic cancer model. Cancer Res; 59: 2635-43 (1999).

5. Behr T, Sgouros G, Stabin M, Behe M, Angerstein C, et al. Studies on the red marrow dosimetry in radioimmunotherapy: an experimental investigation of factors influencing the radiation-induced myelotoxicity in therapy with beta-, Auger/conversion electron-, or alpha-emitters. Clin Cancer Res; 5: 3031s-43s (1999).

6. Kennel S J, Mirzadeh S. Vascular targeted radioimmunotherapy with 213Bi—an alpha-particle emitter. Nucl Med Biol; 25: 241-46 (1998).

7. Zalutsky M, McLendon R, Garg P, Archer G, Schuster J, et al. Radioimmunotherapy of neoplastic meningitis in rats using an alpha-particle-emitting immunoconjugate. Cancer Res; 54: 4719-25 (1994).

8. McDevitt M, Ma D, Lai L, Simon J, Borchardt P, et al. Tumor therapy with targeted atomic nanogenerators. Science; 294: 1537-1540 (2001).

9. Jurcic J G, Larson S M, Sgouros G, et al. Targeted alpha-particle immunotherapy for myeloid leukemia. Blood.; 100: 1233-1239 (2002).

10. Humm J. A microdosimetric model of astatine-211 labeled antibodies for radioimmunotherapy. Int J Radiat Oncol Biol Phys; 13: 1767-73 (1987).

11. Humm J, Chin L. A model of cell inactivation by alpha-particle internal emitters. Radiat Res; 134: 143-50 (1993).

12. Macklis R, Kinsey B, Kassis A, Ferrara J, Archer R, et al. Radioimmunotherapy with alpha-particle-emitting immunoconjugates. Science; 240: 1024-26 (1988).

13. Sgouros S. Long-lived alpha emitters in radioimmunotherapy: the mischievous progeny. Cancer Biother. Biopharm.; 15: 219-221 (2000).

14. Kennel S J, Brechbiel M W, Milenic D E, Schlom J, Mirzadeh S. Actinium-225 conjugates of MAb CC49 and humanized delta CH2CC49. Cancer Biother Radiopharm; 17: 219-31 (2002).

15. Cullis P R, DeKruijff B. Lipid polymorphism and the functional role of lipids in biological membranes. Biochim. Biophys. Acta.; 559: 399-420 (1979).

16. Lasic D D. Novel applications of liposomes. Trends Biotechnol.; 16: 307-321 (1998).

17. Kim, et al. Biochim, Biophys. Acta; 728: 339-348 (1983).

18. Huang. Biochemistry; 8: 334-352 (1969).

19. Kim, et al. Biochim. Biophys. Acta; 646: 1-10 (1981).

20. Bangham, et al. J Mol. Bio.; 13: 238-252 (1965).

21. Jean R. Philippot and Francis Schuber, eds. Liposomes as Tools in Basic Research and Industry. CRC Press; pg.19 (1995).

22. Szoka, et al. Ann. Rev. Biophys. Bioeng.; 9: 465-508 (1980)

23. Kim, et al. Cancer Treat. Rep.; 71: 705-711 (1987).

24. Drummond D D, Meyer O, Hong K, Kirpotin D B, Papahadjopoulos D. Optimizing liposomes for delivery of chemotherapeutic agents to solid tumors. Pharmacol Rev. 51:691-743 (1999).

25. Huang S K, Martin F J, Jay G, Vogel J, Papahadjopoulos, Friend D S. Extravasation and transcytosis of liposomes in Kaposi's sarcoma-like dermal lesions of transgenic mice bearing the HIV tat gene. Am. J. Pathol. 143:10-14 (1993).

26. Wu N Z, Da D, Rudoll T L, Needham D, Whorton A R, Dewhirst M W. Increased microvascular permeability contributes to preferential accumulation of stealth liposomes in tumor tissue. Cancer Res. 53:3765-3770 (1993).

27. Yuan F, Leunig M, Huang S K, Berk D A, Papahadjopoulos D, Jain R K. Microvascular permeability and interstitial penetration of sterically stabilized (stealth) liposomes in a human tumor xenograft. Cancer Res. 54:3352-3356 (1994).

28. Boerman O C, Storm G, Oyen Wv. Sterically stabilized liposomes labelled with In-111 to image focal infection in rats. J Nucl Med. 36:1639-1644 (1995).

29. Emfietzoglou D, Kostarelos K, Sgouros G. An analytic dosimetry study for the use of radionuclide-liposome conjugates in internal radiotherapy. J. Nucl. Med. 42:499-504 (2001).

30. Kennel S J, Chappell L L, Dadachova K, et al. Evaluation of Ac-225 for vascular targeted radioimmunotherapy of lung tumors. Cancer Biother Biopharm. 15:235-244 (2000).

31. Cevc G. The molecular mechanism of interaction between monovalent ions and polar surfaces, such as lipid bilayer membranes. Chem Phys Letters. 170:283-288 (1990).

32. Binder H, Zschoernig O. The effect of metal cations on the phase behavior and hydration characteristics of phospholipid membranes. Chem Phys Lipids. 115:39-61 (2002).

33. Cevc G, Marsh D. Phospholipid bilayers: physical principles and models. New York; 1987

34. Rippe B, Rosengren B I, Venturoli D. The peritoneal microcirculation in peritoneal dialysis. Microcirculation. 8:303-320 (2001).

35. Ahl P L, Bhatia S K, Meers P, et al. Enhancement of the in vivo circulation lifetime of L-a-distearoylphosphatidylcholine liposomes: importance of liposomal aggregation versus complement opsonization. Biochim Biophys Acta. 1329:370-382 (1997).

36. Park J W, Kirpotin D B, Hong K, et al. Tumor targeting using anti-her2 immunoliposomes. J Control Release. 74:95-113 (2001).

37. Brzozowska I, Figaszewski Z A. The equilibrium of phosphatidylcholine-cholesterol in monolayers at the air/water interface. Colloids Surf B 23:51-58 (2002).

38. Castile J D, Taylor K M G. Factors affecting the size distribution of liposomes produced by freeze-thaw extrusion. Int. J. Pharm. 188:87-95 (1999).

39. Stensrud G, Redford K, Smistad G, Karlsen J. Effects of gamma irradiation on solid and lyophilised phospholipids. Rad. Phys. Chem. 56:611-622 (1999).

40. McIntyre J C, Sleight R G. Fluorescence assay for phospholipid membrane asymmetry. Biochemistry. 30:11819-11827 (1991).

41. Provencher S W. A constrained regularization method for inverting data represented by linear algebraic or integral equations. Comput. Phys. Commun. 27:213-227 (1982).

42. Teraoka I. Polymer Solutions: An Introduction to Physical Properties. 1st edition edition: John Wiley & Sons; 2002

43. Hwang, K. J., et al. Encapsulation with High Efficiency of Radioactive Metal Ions in Liposomes. Biochim Biophys Acta. Vol. 716 (1), pg. 101-9 (1982).

44. Ziegler J. Stopping and range of ions in matter (SRIM96). Yorktown, N.Y.: IBM; 1996

45. Ziegler J F, Biersack J P, Littmark U. The stopping and range of ions in solids. New York: Pergamon Press; 1985

46. Maruyama K, Takizawa T, Yuda T, Kennel S J, Huang L, Iwatsuru M. Targetability of novel immunoliposomes modified with amphipathic poly(ethylene glycol)s conjugated at their distal terminals to monoclonal antibodies. Biochim Biophys Acta. 1234:74-80 (1995).

47. Kirpotin D B, Park J W, Hong K, Zalipsky S, Li W-L, Carter P, et al. Sterically stabilized anti-HER2 immunoliposomes: design and targeting to human breast cancer cells in vitro. Biochemistry 36:66-75 (1997).

Any patents or publications mentioned in this specification are indicative of the levels of those skilled in the art to which the invention pertains. These patents and publications are herein incorporated by reference to the same extent as if each individual publication specifically and individually was incorporated by reference.

One skilled in the art will readily appreciate that the present invention is well adapted to carry out the objects and obtain the ends and advantages mentioned, as well as those inherent therein. It will be apparent to those skilled in the art that various modifications and variations can be made in practicing the present invention without departing from the spirit or scope of the invention. Changes therein and other uses will occur to those skilled in the art which are encompassed within the spirit of the invention as defined by the scope of the claims.

What is claimed is:

1. A method of reducing the systemic release of radioactive decay intermediates upon administration of an alpha particle-emitting radionuclide to an individual, comprising the steps of:
incorporating said radionuclide into large liposomes, said liposomes having a diameter sufficient to retain at least a majority of said radioactive decay intermediates; and
administering said large liposomes to said individual, wherein retention within said large liposomes of said radioactive decay intermediates produced by said radionuclide reduces the systemic release thereof.

2. The method of claim 1, further comprising:
entrapping said radionuclide within smaller liposomal vesicles prior to incorporating said radionuclide contained therein into the aqueous phase of said larger liposome.

3. The method of claim 2, further comprising:
labeling said smaller liposomal vesicles with biotin.

4. The method of claim 1, further comprising the step of:
coating outer membrane surfaces of said large liposomes with molecules which preferentially associate with a specific target cell thereby increasing specificity of said large liposomes to said target cell.

5. The method of claim 4, wherein said target cell is a cancer cell, a virally infected cell, an autoimmune cell, or an inflammatory cell.

6. The method of claim 4, wherein said molecules are antibodies, peptides, engineered molecules or fragments thereof.

7. The method of claim 6, wherein at least some of said antibodies are Herceptin.

8. The method of claim 1, further comprising the steps of:
preinjecting the individual with empty large liposomes; and
saturating the reticuloendothelial organs to reduce non-tumor specific spleen and liver uptake of said radionuclide upon adminstration thereof.

9. The method of claim 1, wherein said large liposomes have a diameter of about 600 nm to about 1000 nm.

10. The method of claim 1, wherein said large liposomes comprise molecules incorporated into outer membranes to stabilize said large liposomes.

11. The method of claim 10, wherein said stabilizing molecules are polyethyleneglycol-linked lipids (PEG-lipids).

12. The method of claim 10, wherein said stabilizing molecules further comprise an antibody, peptide, engineered molecule or fragment thereof attached thereto.

13. The method of claim 1, wherein said large liposomes comprise a stabilizing agent incorporated therein or have an aqueous phase with a high pH thereby further facilitating retention of said radioactive decay intermediates.

14. The method of claim 13, wherein said stabilizing agent is a phosphate buffer, insoluble metal binding polymer, resin beads, metal-binding molecules or halogen binding molecules.

15. The method of claim 1, wherein said large liposomes comprise additional molecules, said molecules facilitating membrane fusion with target cells or facilitating endocytosis by target cells.

16. The method of claim 1, wherein said alpha particle emitting radionuclide is incorporated into the aqueous phase as a chelation compound.

17. The method of claim 1, wherein said alpha-particle-emitting radionuclide is ²²⁵Ac, ²²³Ra, ²¹³Bi, or ²¹¹At.

18. The method of claim 1, wherein said alpha particle-emitting radionuclide is a daughter of a beta particle-emitting radionuclide, wherein said beta particle-emitting radionuclide is incorporated within said large liposomes.

19. The method of claim 18, wherein said beta particle-emitting radionuclide is ²¹²Pb.

20. A method of targeting cells in an individual for liposomal delivery of an alpha particle-emitting radionuclide thereto with reduced systemic release of radioactive decay intermediates comprising the steps of:
entrapping said radionuclide within small liposomal vesicles;
incorporating said entrapped radionuclide into the aqueous phase of large liposomes, said liposomes having a diameter sufficient to retain at least a majority of the radioactive decay intermediates of said radionuclide, said liposome comprising:
polyethyleneglycol-linked lipids (PEG-lipids) on outer membranes thereof; and
a targeting agent attached to the PEG-lipids, said targeting agent specific to the cells; and
delivering said radionuclide to the cells whereby said targeting agents target the cells while retention within said large liposomes of said radioactive decay intermediates produced by said radionuclide reduces the systemic release thereof.

21. The method of claim 20, further comprising:
labeling said smaller liposomal vesicles with biotin.

22. The method of claim 20, further comprising the steps of:
preinjecting the individual with empty large liposomes; and
saturating the reticuloendothelial organs to reduce non-tumor specific spleen and liver uptake of said radionuclide upon delivery thereof.

23. The method of claim 20, wherein said large liposomes have a diameter of about 600 nm to about 1000 nm.

24. The method of claim 20, wherein said targeting agents are antibodies, peptides, engineered molecules or fragments thereof.

25. The method of claim 24, wherein at least some of said antibodies are Herceptin.

26. The method of claim 20, wherein said targeted cells are cancer cells, virally infected cells, autoimmune cells, or inflammatory cells.

27. The method of claim 20, wherein said large liposomes further comprise a stabilizing agent incorporated therein or have an aqueous phase with a high pH thereby further facilitating retention of said radioactive decay intermediates.

28. The method of claim 27, wherein said stabilizing agent is a phosphate buffer, insoluble metal binding polymer, resin beads, metal-binding molecules or halogen binding molecules.

29. The method of claim 20, wherein said large liposomes further comprise additional molecules, said molecules facilitating membrane fusion with target cells or facilitating endocytosis by target cells.

30. The method of claim 20, wherein said alpha particle emitting radionuclide is incorporated into the aqueous phase of said small liposomal vesicles as a chelation compound.

31. The method of claim 20, wherein said alpha-particle-emitting radionuclide is ²²⁵Ac, ²²³Ra, ²¹³Bi, or ²¹¹At.

32. The method of claim 20, wherein said alpha particle-emitting radionuclide is a daughter of a beta particle-emitting radionuclide, wherein said beta particle-emitting radionuclide is entrapped within said small liposomal vesicles.

33. The method of claim 32, wherein said beta particle-emitting radionuclide is ²¹²Pb.

34. A method of targeting cancer cells expressing HER-2/neu protein in an individual for liposomal delivery of Ac-225 thereto with reduced systemic release of radioactive decay intermediates thereof comprising the steps of:
entrapping said Ac-225 within small liposomal vesicles;
incorporating said entrapped Ac-225 into the aqueous phase of large liposomes, said liposomes having a diameter sufficient to retain at least a majority of the radioactive decay intermediates of Ac-225, said liposome comprising:
polyethyleneglycol-linked lipids (PEG-lipids) incorporated into outer membranes thereof; and
Herceptin antibodies attached to the PEG-lipids; and
delivering said Ac-225 to the cancer cells whereby said Herceptin targets the HER-2/neu protein expressed on the cells while retention within said large liposomes of said radioactive decay intermediates produced by said radionuclide reduces the systemic release thereof.

35. The method of claim 34, further comprising:
labeling said smaller liposomal vesicles with biotin.

36. The method of claim 34, further comprising the steps of:
preinjecting the individual with empty large liposomes; and
saturating the reticuloendothelial organs to reduce non-tumor specific spleen and liver uptake of said radionuclide upon delivery thereof.

37. The method of claim 34, wherein said large liposomes have a diameter of about 600 nm to about 1000 nm.

38. The method of claim 34, wherein said cancer cells comprise an ovarian carcinoma.

39. The method of claim 34, wherein said large liposomes further comprise a stabilizing agent incorporated therein or have an aqueous phase with a high pH thereby further facilitating retention of said radioactive decay intermediates.

40. The method of claim 39, wherein said stabilizing agent is a phosphate buffer, insoluble metal binding polymer, resin beads, metal-binding molecules or halogen binding molecules.

41. The method of claim 34, wherein said large liposomes further comprise additional molecules, said molecules facilitating membrane fusion with target cells or facilitating endocytosis by target cells.

42. The method of claim 34, wherein said Ac-225 is chelated.

43. An encapsulated alpha particle emitting radionuclide, comprising:
said alpha particle emitting radionuclide;
small liposome vesicles entrapping said alpha particle emitting radionuclide; and
a large liposome incorporating said small liposome vesicles, said large liposome having a diameter sufficient to retain at least a majority of radioactive decay intermediates, said alpha particle emitting radionuclide thereby encapsulated therein.

44. The encapsulated radionuclide of claim 43, further comprising:
labeling said smaller liposomal vesicles with biotin.

45. The encapsulated radionuclide of claim 43, wherein said alpha particle emitting radionuclide is ²²⁵Ac, ²²³Ra, ²¹³Bi, ²¹²Pb, or ²¹¹At.

46. The encapsulated radionuclide of claim 39, wherein said alpha particle-emitting radionuclide is a daughter of a beta particle-emitting radionuclide, wherein said beta particle-emitting radionuclide is encapsulated.

47. The The encapsulated radionuclide of claim 46, wherein said beta particle-emitting radionuclide is ²¹²Pb.

48. The encapsulated radionuclide of claim 43, wherein said radionuclide associates with a membrane of said small liposome or is incorporated into the aqueous compartment of said small liposome as a chelation compound.

49. The method of claim 43, wherein said large liposomes have a diameter of about 600 nm to about 1000 nm.

50. The encapsulated radionuclide of claim 43, wherein said large liposomes further comprise molecules which preferentially associate with a target cell, said molecules coating outer membrane surfaces of said large liposomes.

51. The encapsulated radionuclide of claim 50, wherein said molecules are antibodies, peptides, engineered molecules or fragments thereof.

52. The encapsulated radionuclide of claim 51, wherein at least some of said antibodies are Herceptin.

53. The encapsulated radionuclide of claim 50, wherein said target cell is a cancer cell, a virally infected cell, an autoimmune cell, or an inflammatory cell.

54. The encapsulated radionuclide of claim 43, wherein said large liposomes further comprise molecules incorporated into outer membranes to stabilize said large liposomes.

55. The method of claim 54, wherein said stabilizing molecules further comprise an antibody, peptide, engineered molecule or fragment thereof attached thereto.

56. The encapsulated radionuclide of claim 54, wherein said stabilizing molecules are polyethyleneglycol-linked lipids (PEG-lipids).

57. The encapsulated radionuclide of claim 43, wherein said large liposomes comprise a stabilizing agent incorporated therein or have an aqueous phase with a high pH.

58. The encapsulated radionuclide of claim 57, wherein said stabilizing agent is a phosphate buffer, insoluble metal binding polymer, resin beads, metal-binding molecules or halogen binding molecules.

59. The encapsulated radionuclide of claim 43, wherein said large liposomes comprise molecules facilitating membrane fusion with a target cell or facilitating endocytosis by a target cell.
